(19) United States
(12) Patent Application Publication
(10) Pub. No.: US 2020/0319188 A1
(43) Pub. Date: Oct. 8, 2020

(54) METHODS OF PRODUCING CIRCULATING ANALYTE PROFILES AND DEVICES FOR PRACTICING SAME

(71) Applicant: MagArray, Inc., Milpitas, CA (US)

(72) Inventors: Shan Xiang Wang, Palo Alto, CA (US); Chih-Yin Juang, Menlo Park, CA (US); Heng Yu, Campbell, CA (US); Michael J. Beggs, San Jose, CA (US); Luis Carbonell, Huntington Beach, CA (US)

(21) Appl. No.: 16/811,518

(22) Filed: Mar. 6, 2020

Related U.S. Application Data
(60) Provisional application No. 62/829,245, filed on Apr. 4, 2019.

(51) Int. Cl. G01N 33/574; G01N 33/543

(52) U.S. Cl. CPC G01N 33/57423; G01N 33/54326; G01N 2333/8146; G01N 2333/71; G01N 2333/521

(57) ABSTRACT

Aspects of the present disclosure include methods of producing a circulating analyte profile of a subject. The methods include contacting a blood sample from a subject with a panel of probes for specific binding to analytes, and detecting the presence or absence of binding of the analytes to probes of the panel of probes. Also provided are sensor devices including a panel of capture probes and useful, e.g., for practicing the methods of the present disclosure.

### CROSS-REFERENCE TO RELATED APPLICATIONS

This application claims the benefit of U.S. Provisional Application No. 62/829,245, filed Apr. 4, 2019, the disclosure of which is incorporated herein by reference.

### INTRODUCTION

Lung cancer remains the most lethal and second most prevalent cancer in the United States with a 5 to 15% five-year survival rate for advanced stage IV non-small cell lung cancer (NSCLC). NSCLC is the most common type of lung cancer, and when caught early while still in stage I, the five-year survival rate is almost 80%. Detecting early disease has therefore been the focus of intense investigation and public health programs aimed at identifying who is at risk of having lung cancer are underway. The number one risk for lung cancer continues to be smoking with the CDC reporting that up 90% of lung cancer deaths are linked to cigarette smoking. The U.S. Preventive Services Task Force (USPSTF) has issued recommendations for annual lung cancer screening with low-dose computed tomography (LDCT) for adults aged 55 to 80 years with a history of smoking 30 pack-years of cigarettes, whether they are current smokers or former smokers having quit in the past 15 years. Consequently, an increasing number of individuals are undergoing annual LDCT screening for evidence of a lung nodule, which is the first indication of lung cancer. The appearance of a nodule on an LDCT scan is not proof of lung cancer because non-cancerous nodules also occur in the lung, and at a much greater frequency than do cancerous nodules. Up to 94% of the lung nodules found on LDCT scans are due to benign disease. Such a high rate of false positive results is subjecting hundreds of thousands of individuals to unnecessary interventions and invasive procedures that can not only cause significant harm but also place a significant burden on an already over-taxed health care system. A need exists for an effective non-invasive method to assess whether a lung nodule detected by LDCT is a cancerous or benign lesion.

### SUMMARY

Aspects of the present disclosure include methods of producing a circulating analyte profile of a subject. The methods include contacting a blood sample from a subject with a panel of probes for specific binding to analytes, and detecting the presence or absence of binding of the analytes to probes of the panel of probes. In some embodiments, the panel of probes includes probes for specific binding to analytes including two, three or each of carcinoembryonic antigen (CEA), C-X-C motif chemokine ligand 4 (CXCL4), C-X-C motif chemokine ligand 7 (CXCL7), and C-X-C motif chemokine ligand 10 (CXCL10). In some embodiments, such a panel of probes further includes probes for specific binding to two or each of epidermal growth factor receptor (EGFR), pro-surfactant protein B (pro-SFTPB), and tissue inhibitor of metalloproteinase 1 (TIMP1). Also provided are sensor devices (e.g., magnetic sensor devices) including a panel of capture probes and useful, e.g., for practicing the methods of the present disclosure.

### BRIEF DESCRIPTION OF THE FIGURES

FIG. 1 Distributions of biomarkers in 405 samples stratified by smoking history.

FIG. 2 ROC curve of Model 217_3092 trained with a Former smokers 1/3 subset and tested on a 2/3 subset compared to the Mayo model ROC curve.

FIG. 3 ROC curve of Model 217_3092 trained with the former smokers 1/3 subset and tested on the Mayo Model intermediate risk (IR) subjects in the 2/3 subset compared to the Mayo model ROC curve.

FIG. 4 ROC curve of Model 217_3092 trained with the Current smokers 2/3 subset and tested on the 1/3 subset compared to the Mayo model ROC curve.

FIG. 5 ROC curve of Model 217_3092 trained with the Current smokers 2/3 subset and tested on the Mayo Model intermediate risk (IR) subjects in the 1/3 subset compared to the Mayo model ROC curve.

### DETAILED DESCRIPTION

Aspects of the present disclosure include methods of producing a circulating analyte profile of a subject. The methods include contacting a blood sample from a subject with a panel of probes for specific binding to analytes, and detecting the presence or absence of binding of the analytes to probes of the panel of probes. Also provided are sensor devices including a panel of capture probes and useful, e.g., for practicing the methods of the present disclosure.

Before the methods, devices and kits of the present disclosure are described in greater detail, it is to be understood that the methods, devices and kits are not limited to particular embodiments described, as such may, of course, vary. It is also to be understood that the terminology used herein is for the purpose of describing particular embodiments only, and is not intended to be limiting, since the scope of the methods, devices and kits will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the methods, devices and kits. The upper and lower limits of these smaller ranges may independently be included in the smaller ranges and are also encompassed within the methods, devices and kits, subject to any specifically excluded limit in the stated range. Where the stated range includes one or both of the limits, ranges excluding either or both of those included limits are also included in the methods, devices and kits.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the methods, devices and kits belong. Although any methods, devices and kits similar or equivalent to those described herein can also be used in the practice or testing of the methods, devices and kits, representative illustrative methods, devices and kits are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present methods, devices and kits are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the methods, devices and kits, which are, for clarity, described in the context of separate embodiments, may also be provided in combination in a single embodiment. Conversely, various features of the methods, devices and kits, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present methods, devices and kits and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

Methods

Aspects of the present disclosure include methods of producing a circulating analyte profile of a subject. The methods include contacting a blood sample from a subject with a panel of probes for specific binding to analytes, and detecting the presence or absence of binding of the analytes to probes of the panel of probes. In certain aspects, the detecting includes quantifying detected analytes.

A probe of the panel of probes can be any molecule that specifically binds to an analyte of interest. Analytes of interest include, but are not limited to, proteins (including non-antibody proteins, antibody proteins, etc.), nucleic acids (e.g., tumor DNA or RNA), and cells, e.g., circulating tumor cells. The probes of the panel of probes may be selected depending upon the nature of the analytes to be detected. For example, if one of the two or more analytes is a protein (e.g., a non-antibody protein or antibody protein), an antibody, ligand, or the like that specifically binds that protein may be employed as a probe in the panel of probes. If one of the two or more analytes is an antibody, the corresponding antigen for that antibody may be employed as a probe in the panel of probes, or an antibody that binds to the antibody may be employed. If one of the two or more analytes is a nucleic acid, a nucleic acid sufficiently complementary to a unique region of that nucleic acid to achieve specific binding under the desired contacting conditions may be employed as a probe in the panel of probes, for example. Proteins (e.g., nucleic acid binding proteins, antibodies, and the like) may also be employed for binding to nucleic acid analytes.

The term “binding” refers to a direct association between two molecules, due to, for example, covalent, electrostatic, hydrophobic, ionic and/or hydrogen-bond interactions. The probes of the panel of probes bind specifically to their corresponding analytes. Non-specific binding (NSB) typically refers to the binding of an antibody to something other than its homologous antigen such as various other antigens in the sample. Under certain assay conditions, NSB would refer to binding with an affinity of less than about 10⁻⁷ M, e.g., binding with an affinity of 10⁻⁶ M, 10⁻⁵ M, 10⁻⁴ M, etc.

By “specifically binds” or “specific binding” is meant a probe binds to its corresponding analyte with an affinity or Kₐ (that is, an equilibrium association constant of a particular binding interaction with units of 1/M) of, for example, greater than or equal to about 10⁵ M⁻¹. In certain embodiments, the extracellular binding domain binds to an antigen with a Kₐ greater than or equal to about 10⁶ M⁻¹, 10⁷ M⁻¹, 10⁸ M⁻¹, 10⁹ M⁻¹, 10¹⁰ M⁻¹, 10¹¹ M⁻¹, 10¹² M⁻¹, or 10¹³ M⁻¹. “High affinity” binding refers to binding with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, at least 10¹³ M⁻¹, or greater. Alternatively, affinity may be defined as an equilibrium dissociation constant (K_{D}) of a particular binding interaction with units of M (e.g., 10⁻⁵ M to 10⁻¹³ M, or less). In some embodiments, specific binding means the extracellular binding domain binds to the target molecule with a K_{D} of less than or equal to about 10⁻⁵ M, less than or equal to about 10⁻⁶ M, less than or equal to about 10⁻⁷ M, less than or equal to about 10⁻⁸ M, or less than or equal to about 10⁻⁹ M, 10⁻¹⁰ M, 10⁻¹¹ M, or 10⁻¹² M or less. The binding affinity of a probe for its target analyte can be readily determined using conventional techniques, e.g., by competitive ELISA (enzyme-linked immunosorbent assay), equilibrium dialysis, by using surface plasmon resonance (SPR) technology (e.g., the BIAcore 2000 instrument, using general procedures outlined by the manufacturer); by radioimmunoassay; or the like.

The panel of probes includes a suitable number of probes for specific binding to the number of unique circulating analytes of interest. According to certain embodiments, the panel of probes includes a suitable number of probes for specific binding to from 4 to 5 analytes, from 6 to 10 analytes, from 10 to 15 analytes, from 15 to 20 analytes, from 20 to 25 analytes, from 25 to 30 analytes, from 30 to 35 analytes, from 35 to 40 analytes, from 40 to 45 analytes, from 45 to 50 analytes, from 50 to 60 analytes, from 60 to 70 analytes, from 70 to 80 analytes, from 80 to 90 analytes, from 90 to 100 analytes, from 100-200 analytes, from 200 to 300 analytes, from 300 to 400 analytes, from 400 to 500 analytes, or from 500 to 1000 analytes.

In certain embodiments, the panel of probes includes probes for specific binding to 4 or more, 5 or more, 6 or more, 7 or more, 8 or more, 9 or more, 10 or more, 15 or more, 20 or more, or 25 or more analytes. According to some embodiments, the panel of probes includes probes for specific binding to 200 or fewer analytes, 150 or fewer analytes, 125 or fewer analytes, 100 or fewer analytes, 75 or fewer analytes, 50 or fewer analytes, 40 or fewer analytes, 30 or fewer analytes, 25 or fewer analytes, 20 or fewer analytes, 15 or fewer analytes, or 10 or fewer analytes.

According to some embodiments, the panel of probes includes probes for specific binding to two or more (e.g., 3 or more, 4 or more, 5 or more, 6 or more, 7 or more, 8 or more, 9 or more, or 10 or more) of carcinoembryonic antigen (CEA), C-X-C motif chemokine ligand 4 (CXCL4—also known as platelet factor 4 (or PF4)), C-X-C motif chemokine ligand 7 (CXCL7—also known as neutrophil activating protein 2 (or NAP2)), C-X-C motif chemokine ligand 10 (CXCL10—also known as interferon gamma-induced protein 10 (or IPI 0)), epidermal growth factor receptor (EGFR), pro-surfactant protein B (pro-SFTPB), tissue inhibitor of metalloproteinase 1 (TIMP1), anti-angiopoietin-like protein 3 antibody (anti-ANGPTL3), anti-14-3-3 protein theta antibody (anti-YWHAQ), anti-laminin alpha 1 antibody (anti-LAMR1), human epididymis protein 4 (HE4), anterior gradient protein 2 (AGR2), chromogranin A (CHGA), leucine-rich alpha-2-glycoprotein 1 (LRG1), anti-annexin 1 antibody (anti-ANXA1), anti-ubiquilin 1 antibody (anti-UBQLN1), interleukin 6 (IL6), interleukin 8 (IL8), C-X-C motif chemokine ligand 2 (CXCL2), C-X-C motif chemokine ligand 12 (CXCL1 2), C-X-C motif chemokine ligand 14 (CXCL14), defensin, beta 1 (DEFB1), fibroblast growth factor 2 (FGF2), cluster of differentiation 97 (CD97), pro-platelet basic protein (PPBP), procalcitonin (PCT), receptor for advanced glycation end products (RAGE), S100 calcium-binding protein A4 (S100A4), S100 calcium-binding protein A8 (S100A8), and osteopontin (OPN), in any desired combination.

In certain embodiments, the panel of probes includes probes for specific binding to one, two, three, or each of CEA, CXCL4, CXCL7, and CXCL10, in any desired combination. According to some embodiments, such a panel of probes further includes probes for specific binding to one, two, or each of EGFR, pro-SFTPB, and TIMP1, in any desired combination. In certain embodiments, such a panel of probes further includes one or more probes for specific binding to one or any combination of additional analytes selected from anti-ANGPTL3, anti-YWHAQ, anti-LAMR1, HE4, AGR2, CHGA, LRG1, anti-ANXA1, anti-UBQLN1, IL6, IL8, CXCL2, CXCL12, CXCL14, DEFB1, FGF2, CD97, PPBP, PCT, RAGE, S100A4, S100A8, and OPN, in any desired combination, where the method further includes detecting the presence or absence of binding of the one or any combination of additional analytes to probes of the panel of probes to produce the circulating analyte profile of the subject.

According to some embodiments, the panel of probes includes one or more probes for binding to one or more types of circulating cells. Circulating cells of interest include, but are not limited to, circulating tumor cells and circulating stem cells. By “circulating tumor cell” (CTC) is meant a cancer cell that is exfoliated from a solid tumor of a subject and is found in the subject's circulation, e.g., the subject's peripheral blood, bone marrow, and/or the like. A probe may bind to a circulating cell (e.g., a CTC) by virtue of the probe having specificity for a known cell surface molecule (e.g., a receptor, adhesion molecule, etc.) expressed by the circulating cell of interest. When the circulating cell is a CTC, the probe (e.g., an antibody probe) may specifically bind to a tumor-associated or tumor-specific antigen expressed by the CTC. By “tumor-associated antigen” is meant a cell surface molecule expressed on malignant cells with limited expression on cells of normal tissues, or a cell surface molecule expressed at much higher density on malignant versus normal cells. A “tumor-specific antigen” is an antigen present on the surface of malignant cells and not present on non-malignant cells. The types of CTCs that may be bound by probes of the panel of the probes may vary, e.g., depending on the type of solid tumor from which the CTC sloughed off. In certain aspects, the panel of the probes may include probes for specific binding to CTCs, which probes specifically bind to epithelial cell adhesion molecule (EpCAM) and/or any other useful cell surface CTC molecules. As such, in some embodiments, the panel of probes further includes probes for binding to circulating tumor cells, where the method further includes detecting the presence or absence of binding of the circulating tumor cells to probes of the panel of probes to produce the circulating analyte profile of the subject. In certain embodiments, detecting the presence or absence of binding of the circulating tumor cells includes quantifying detected circulating tumor cells.

According to certain embodiments, the panel of probes includes one or more probes for binding to one or more types of circulating nucleic acids. Circulating nucleic acids of interest include circulating double or single-stranded DNA, circulating double or single-stranded RNA, circulating DNA-RNA hybrids, etc. In certain aspects, the panel includes one or more probes for specific binding to one or more circulating tumor DNAs (ctDNA). Dying tumor cells release small pieces of their DNA into the bloodstream, and the amount/concentration of ctDNA in blood often increases as the cancer stage increases. According to certain embodiments, the panel of probes includes a probe for specific binding to a ctDNA that includes a somatic mutation known to be associated with (or specific to) a tumor type of interest. Clinically relevant ctDNAs include those described in Bettegowda et al. (2014) Sci. TransL Med. 6(224): 224ra24. As such, in some embodiments, the panel of probes further includes probes for binding to tumor DNA, where the method further includes detecting the presence or absence of binding of tumor DNA to probes of the panel of probes to produce the circulating analyte profile of the subject. In certain embodiments, detecting the presence or absence of binding of tumor DNA includes quantifying detected tumor DNA.

The methods of the present disclosure include detecting the presence or absence of binding of analytes to probes of the panel of probes, to produce a circulating analyte profile of the subject. In certain aspects, the detecting includes quantifying detected analytes. Any of a variety of suitable assay formats and detection approaches may be employed. In certain aspects, the probes of the panel of probes may be attached directly or indirectly to a solid support, such as a bead (e.g., a microparticle, nanoparticle, or the like) or a substantially flat solid support/substrate. According to certain embodiments, the probes may be attached to a solid support as an array. For example, the panel of probes may be a panel of probes provided as an addressable probe array.

In certain aspects, detecting the presence or absence of binding of analytes of the two or more analytes to probes of the panel of probes is carried out using a sandwich assay. For example, the probes of the panel of probes may be attached to a solid surface (e.g., as an array) for capturing the analytes, and detection reagents are added that bind (e.g., specifically bind) to the analytes (if present in the blood sample) at sites of the analytes not bound by the probes. In certain aspects, a detection reagent is a detection antibody that binds to an epitope of the analyte that is different from the binding site (e.g., epitope) to which the probe of the panel of probes binds. As a result, the analyte is “sandwiched” between the probe and the detection reagent. The detection reagents may include detectable labels such that detecting the presence or absence of binding of analytes of the two or more analytes to probes of the panel of probes involves detecting the labels of the detection reagents. According to certain embodiments, a secondary detection reagent is employed. Suitable secondary reagents include labeled secondary antibodies (e.g., fluorescently labeled antibodies, magnetic labeled antibodies, etc.), secondary antibodies linked to an enzyme that catalyzes the conversion of a substrate to a detectable product, and the like. Additional details and design considerations for sandwich and other assays that find use in practicing the methods of the present disclosure are described, e.g., in Cox et al. (2014) Immunoassay Methods, Eli Lilly & Company and the National Center for Advancing Translational Sciences.

In certain aspects, a detection reagent that binds to the analyte bound by the probe is an antibody. Such a detection reagent may be a modified antibody. The modified antibody may be configured to specifically bind to the analyte of interest and may also include one or more additional members of a specific binding pair. The one or more members of a specific binding pair may be configured to specifically bind to a complementary member of the specific binding pair. In certain instances, the complementary member of the specific binding pair is bound to a magnetic label, e.g., when a magnetic sensor device is employed to carry out the method. An antibody detection reagent may be modified to include biotin, which biotin will specifically bind to streptavidin, e.g., a magnetic label modified to include streptavidin. As such, in certain aspects, the detection reagent specifically binds to the analyte (e.g., through an antibody-antigen interaction) and specifically binds to a label (e.g., a magnetic label) via a selected interaction (e.g., through a streptavidin-biotin interaction). The detection reagent may be configured to bind to the analyte and a label (e.g., a magnetic label). Stated another way, the detection reagent may be configured such that specific binding of the analyte to the detection reagent does not significantly interfere with the ability of the detection reagent to specifically bind to a label. Similarly, the detection reagent may be configured such that specific binding of the label to the detection reagent does not significantly interfere with the ability of the detection reagent to bind to the analyte.

Analytes in the blood sample may be determined qualitatively or quantitatively. Qualitative determination includes determinations in which a simple yes/no result with respect to the presence of an analyte in the sample is provided to a user. Quantitative determination includes both semi-quantitative determinations in which a rough scale result, e.g., low, medium, high, is provided to a user regarding the amount of analyte in the sample and fine scale results in which an precise measurement of the concentration of the analyte is provided to the user.

The circulating analyte profile may be produced from a blood sample (e.g., a whole blood sample, a plasma sample, or a serum sample) obtained from any of a variety of subjects. Generally, such subjects are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some embodiments, the circulating analyte profile is produced from a blood sample obtained from a human subject.

According to some embodiments, the subject for which the circulating analyte profile is produced is from a population having a high risk of lung cancer. A subject may be at a high risk for lung cancer due to a variety of genetic, behavioral and/or environmental factors. According to certain embodiments, the subject is from a population having a high risk of lung cancer due to the subject being a former smoker (e.g., a past heavy smoker) or a current smoker. By “former smoker” is meant the subject is not a smoker at the time the blood sample for use in the method is obtained from the subject. My “current smoker” is meant the subject is a smoker at the time the blood sample for use in the method is obtained from the subject. According to certain embodiments, the subject being from a population having a high risk of lung cancer means the subject is from 55 to 74 years of age, has a minimum smoking history of 30 pack-years or more (where a “pack-year” is equal to the number of cigarette packs smoked per day×the number of years smoked), currently smokes or quit smoking within the past 15 years, and are apparently disease-free at the time the circulating analyte profile is produced. For example, a past heavy smoker may have a smoking history of 30 pack-years or more.

In certain aspects, the subject for which the circulating analyte profile is produced has a lung nodule (or “lesion”), e.g., an indeterminate lung nodule/lesion. In some instances, an indeterminate lung nodule is identified/detected by low-dose computed tomography (LDCT), chest x-ray, CT scan of the chest, MRI of the chest, positron emission tomography (PET) scan of the chest, or other suitable imaging approach. The indeterminate nodule may be benign (non-cancer) and caused by scarring, inflammation, infection, or the like. In other instances, the nodule may be malignant, e.g., a lung cancer (e.g., an early lung cancer) or a cancer that has spread to the lung from another cancer in the body. As described further herein and demonstrated in the Experimental section below, the circulating analyte profile of the subject may form the basis (e.g., complete or partial basis) for assessing the risk of the lung nodule being malignant.

Magnetic Sensor-Based Methods

According to certain embodiments, the methods of the present disclosure are carried out using a magnetic sensor device. For example, the panel of probes may be arrayed (e.g., provided as an addressable probe array) on a magnetic sensor chip of a magnetic sensor device. The magnetic sensor device may have two or more magnetic sensors having panels of probes (e.g., identical or different arrays of capture probes) attached to the surface thereof. Any of the panels of probes described above may be employed. In certain aspects, each of the two or more magnetic sensors having panels of capture probes attached to the surface thereof includes capture probes for binding to the same circulating analytes.

Methods of the present disclosure that employ a magnetic sensor device may include contacting the magnetic sensor device having the panel of capture probes attached to the surface thereof (e.g., arrayed) with the blood sample and detecting signals indicating the binding of the analytes (if present in the blood sample) to the panel of capture probes. In some cases, the magnetic sensor device includes sensors configured to detect the presence of nearby magnetic labels without any direct physical contact between the magnetic sensor and a magnetic label. A magnetic label may be bound, either directly or indirectly, to an analyte, which in turn may be bound, either directly or indirectly, to the magnetic sensor. If the bound magnetic label is positioned within the detection range of the magnetic sensor, then the magnetic sensor may provide a signal indicating the presence of the bound magnetic label, and thus indicating the presence of the analyte.

In certain aspects, the methods of the present disclosure are performed using a sandwich assay in which the panel of probes is attached to a surface of a sensing region of the magnetic sensor device. The blood sample is dispensed on the sensing region to contact the blood sample with the panel of probes under conditions in which analytes of the two or more analytes (if present in the blood sample) bind to their respective probes. With or without washing, detection reagents may be added that bind to analytes of the two or more analytes which are bound to the probes of the panel of probes. In some instances, the detection reagents are directly bound to a magnetic label. In other aspects, the detection reagents are not directly bound to a magnetic label, but rather secondary magnetically labeled detection reagents that bind to the detection reagents are employed. For example, a detection reagent may specifically bind to the analyte (e.g., through an antibody-antigen interaction) and specifically bind to a magnetic label via a selected interaction (e.g., through a streptavidin-biotin interaction). Binding of the detection reagent(s) to a surface-bound analyte positions the magnetic label within the detection range of the magnetic sensor, such that a detectable signal indicative of the presence of the analyte is induced in the magnetic sensor.

In certain embodiments, an electrical signal is generated in response to a magnetic label in proximity to a surface of the magnetic sensor. For example, the magnetic sensor may be configured to detect changes in the resistance of the magnetic sensor induced by changes in the local magnetic field. In some cases, binding of a magnetic label (e.g., a magnetic nanoparticle label) in close proximity to the magnetic sensor, induces a detectable change in the resistance of the magnetic sensor. For instance, in the presence of an applied external magnetic field, the magnetic labels near the magnetic sensor may be magnetized. The local magnetic field of the magnetized magnetic labels may induce a detectable change in the resistance of the underlying magnetic sensor. Thus, the presence of the magnetic labels can be detected by detecting changes in the resistance of the magnetic sensor. As will be described in further detail below, a magnetic sensor device that finds use in practicing the methods of the present disclosure may include a magnetoresistive element. Non-limiting examples of magnetoresistive elements which may be employed include spin valve magnetoresistive elements and magnetic tunnel junction (MTJ) magnetoresistive elements.

In some instances, the methods are wash-free methods of evaluating the presence of the analytes in the blood sample. By “wash-free” is meant that no washing step is performed following reagent and/or blood sample contact with a magnetic sensor. As such, no step is performed during the assays of these embodiments in which unbound reagent (e.g., unbound magnetic labels) or unbound sample is removed from the magnetic sensor surface. Accordingly, while the methods may include sequential contact of one or more distinct reagents and/or samples to a magnetic sensor surface, at no point during the assay is the sample surface contacted with a fluid in a manner that removes unbound reagent or sample from the magnetic sensor surface. For example, in certain embodiments, no washing step is performed following contact of the magnetic sensor surface with the blood sample. In some cases, the method does not include a washing step following contact of the magnetic sensor surface with a magnetic label. In certain instances, no washing step is performed following contact of the magnetic sensor surface with a detection reagent.

In certain embodiments where a wash step is performed, the wash step does not substantially change the signals from the magnetic sensor. The wash step may not result in a substantial change in the signals from the magnetic sensor because, in some instances, unbound magnetic labels do not have a substantially detectable signal as described herein. For example, if a wash step is performed, in some cases, the wash step results in a signal change of 25% or less, such as 20% or less, or 15% or less, or 10% or less, or 5% or less, or 4% or less, or 3% or less, or 2% or less, or 1% or less, as compared to a signal obtained prior to the wash step. In some embodiments, the wash step results in a decrease in the signals from the magnetic sensor of 25% or less, such as 20% or less, or 15% or less, or 10% or less, or 5% or less, or 4% or less, or 3% or less, or 2% or less, or 1% or less.

Embodiments of the methods may also include obtaining a real-time signal from the magnetic sensor device. By “real-time” is meant that a signal is observed as it is being produced. For example, a real-time signal is obtained from the moment of its initiation and is obtained continuously over a given period of time. Accordingly, certain embodiments include observing the evolution in real time of the signal associated with the occurrence of a binding interaction of interest (e.g., the binding of analytes of the two or more analytes of interest to the magnetic sensor and/or binding of a magnetic label to the analyte of interest). The real-time signal may include two or more data points obtained over a given period of time, where in certain embodiments the signal obtained is a continuous set of data points (e.g., in the form of a trace) obtained continuously over a given period of time of interest. The time period of interest may vary, ranging in some instances from 0.5 min to 60 min, such as 1 min to 30 min, including 1 min to 15 min, or 1 min to 10 min. For example, the time period may begin at the moment of initiation of the real-time signal and may continue until the sensor reaches a maximum or saturation level (e.g., where all the analyte binding sites on the sensor are occupied). For example, in some cases, the time period begins when the blood sample is contacted with the sensor. In some cases, the time period may begin prior to contacting the blood sample with the sensor, e.g., to record a baseline signal before contacting sample to the sensor. The number of data points in the signal may also vary, where in some instances, the number of data points is sufficient to provide a continuous stretch of data over the time course of the real-time signal. By “continuous” is meant that data points are obtained repeatedly with a repetition rate of 1 data point per minute or more, such as 2 data points per minute or more, including 5 data points per minute or more, or 10 data points per minute or more, or 30 data points per minute or more, or 60 data points per minute or more (e.g., 1 data point per second or more), or 2 data points per second or more, or 5 data points per second or more, or 10 data points per second or more, or 20 data points per second or more, or 50 data points per second or more, or 75 data points per second or more, or 100 data points per second or more.

A real-time signal may be a real-time analyte-specific signal. A real-time analyte-specific signal is a real-time signal as described above that is obtained only from a specific analyte of the two or more analytes of interest. In these embodiments, unbound analytes and unbound magnetic labels do not produce a detectable signal. As such, the real-time signal that is obtained is only from the specific magnetically-labeled analyte of interest bound to the magnetic sensor and substantially no signal is obtained from unbound magnetic labels or other reagents (e.g., analytes not specifically bound to the sensor).

In some embodiments, the signal is observed while the assay device is in a wet condition. By “wet” or “wet condition” is meant that the assay composition (e.g., an assay composition that includes the blood sample, a magnetic label, and one or more detection reagents) is still in contact with the surface of the magnetic sensor. As such, there is no need to perform any washing steps to remove the non-binding moieties that are not of interest or the excess unbound magnetic labels or capture probes. In certain embodiments, the use of magnetic labels and magnetic sensors, as described above, facilitates “wet” detection because the signal induced in the magnetic sensor by the magnetic label decreases as the distance between the magnetic label and the surface of the magnetic sensor increases. For example, the use of magnetic labels and magnetic sensors, as described above, may facilitate “wet” detection because the magnetic field generated by the magnetic labels decreases as the distance between the magnetic label and the surface of the magnetic sensor increases. In some instances, the magnetic field of the magnetic label bound to the surface-bound analyte significantly exceeds the magnetic field from the unbound magnetic labels dispersed in solution. For example, as described above, a real-time analyte-specific signal may be obtained only from the specific magnetically-labeled analyte of interest bound to the magnetic sensor and substantially no signal may be obtained from unbound magnetic labels dispersed in solution (e.g., not specifically bound to the sensor). The unbound magnetic labels dispersed in solution may be at a greater distance from the surface of the magnetic sensor and may be in Brownian motion, which may reduce the ability of the unbound magnetic labels to induce a detectable change in the resistance of the magnetic sensor. Unbound magnetic labels may also be suspended in solution, for example as a colloidal suspension (e.g., due to having a nanometer-scale size), which may reduce the ability of the unbound magnetic labels to induce a detectable change in the resistance of the magnetic sensor.

Magnetic labels that may be employed in various methods (e.g., as described herein) may vary, and include any type of label that induces a detectable signal in a magnetic sensor when the magnetic label is positioned near the surface of the magnetic sensor. Magnetic labels are labeling moieties that, when sufficiently associated with a magnetic sensor, are detectable by the magnetic sensor and cause the magnetic sensor to output a signal. For example, the presence of a magnetic label near the surface of a magnetic sensor may induce a detectable change in the magnetic sensor, such as, but not limited to, a change in resistance, conductance, inductance, impedance, etc. In some cases, the presence of a magnetic label near the surface of a magnetic sensor induces a detectable change in the resistance of the magnetic sensor. Magnetic labels of interest may be sufficiently associated with a magnetic sensor if the distance between the center of the magnetic label and the surface of the sensor is 1000 nm or less, such as 800 nm or less, such as 400 nm or less, including 100 nm or less, or 75 nm or less, or 50 nm or less, or 25 nm or less, or 10 nm or less.

In certain instances, the magnetic labels include one or more materials selected from paramagnetic, superparamagnetic, ferromagnetic, ferrimagnetic, anti-ferromagnetic materials, combinations thereof, and the like. For example, the magnetic labels may include superparamagnetic materials. In certain embodiments, the magnetic labels are configured to be nonmagnetic in the absence of an external magnetic field. By “nonmagnetic” is meant that the magnetization of a magnetic label is zero or averages to zero over a certain period of time. In some cases, the magnetic label may be nonmagnetic due to random flipping of the magnetization of the magnetic label over time. Magnetic labels that are configured to be nonmagnetic in the absence of an external magnetic field may facilitate the dispersion of the magnetic labels in solution because nonmagnetic labels do not normally agglomerate in the absence of an external magnetic field or even in the presence of a small magnetic field in which thermal energy is still dominant. In certain embodiments, the magnetic labels include superparamagnetic materials or synthetic antiferromagnetic materials. For instance, the magnetic labels may include two or more layers of antiferromagnetically-coupled ferromagnets.

In certain embodiments, the magnetic labels are high moment magnetic labels. The magnetic moment of a magnetic label is a measure of its tendency to align with an external magnetic field. By “high moment” is meant that the magnetic labels have a greater tendency to align with an external magnetic field. Magnetic labels with a high magnetic moment may facilitate the detection of the presence of the magnetic labels near the surface of the magnetic sensor because it is easier to induce the magnetization of the magnetic labels with an external magnetic field.

In certain embodiments, the magnetic labels include, but are not limited to, Co, Co alloys, ferrites, cobalt nitride, cobalt oxide, Co—Pd, Co—Pt, iron, iron oxides, iron alloys, Fe—Au, Fe—Cr, Fe—N, Fe₃O₄, Fe—Pd, Fe—Pt, Fe—Zr—Nb—B, Mn—N, Nd—Fe—B, Nd— Fe—B—Nb—Cu, Ni, Ni alloys, combinations thereof, and the like. Examples of high moment magnetic labels include, but are not limited to, Co, Fe or CoFe nanocrystals, which may be superparamagnetic at room temperature, and synthetic antiferromagnetic nanoparticles.

In some embodiments, the surface of the magnetic label is modified. In certain instances, the magnetic labels may be coated with a layer configured to facilitate stable association of the magnetic label with one member of a binding pair, as described above. For example, the magnetic label may be coated with a layer of gold, a layer of poly-L-lysine modified glass, dextran, and the like. In certain embodiments, the magnetic labels include one or more iron oxide cores imbedded in a dextran polymer. Additionally, the surface of the magnetic label may be modified with one or more surfactants. In some cases, the surfactants facilitate an increase in the water solubility of the magnetic labels. In certain embodiments, the surface of the magnetic labels is modified with a passivation layer. The passivation layer may facilitate the chemical stability of the magnetic labels in the assay conditions. For example, the magnetic labels may be coated with a passivation layer that includes gold, iron oxide, polymers (e.g., polymethylmethacrylate films), and the like.

In certain embodiments, the magnetic labels have a spherical shape. Alternatively, the magnetic labels can be disks, rods, coils, or fibers. In some cases, the size of the magnetic labels is such that the magnetic labels do not interfere with the binding interaction of interest. For example, the magnetic labels may be comparable to the size of the analyte and the capture probe, such that the magnetic labels do not interfere with the binding of the capture probe to the analyte. In some cases, the magnetic labels are magnetic nanoparticles, or contain multiple magnetic nanoparticles held together by a suitable binding agent. In some embodiments, the average diameter of the magnetic labels is from 5 nm to 250 nm, such as from 5 nm to 150 nm, including from 10 nm to 100 nm, for example from 25 nm to 75 nm. For example, magnetic labels having an average diameter of 5 nm, 10 nm, 20 nm, 25 nm, 30 nm, 35 nm, 40 nm, 45 nm, 50 nm, 55 nm, 60 nm, 70 nm, 80 nm, 90 nm, or 100 nm, as well as magnetic labels having average diameters in ranges between any two of these values, may be used with the subject methods. In some instances, the magnetic labels have an average diameter of 50 nm.

Magnetic labels and their conjugation to biomolecules are further described in U.S. Pat. No. 9,863,939 entitled “Analyte Detection with Magnetic Sensors”, the disclosure of which is hereby incorporated herein by reference in its entirety for all purposes.

Risk Assessment

The methods of the present disclosure may further include assessing the risk that the subject has a disease or condition based on the circulating analyte profile. By way of example, as described above and demonstrated in the Experimental section below, the subject for which the circulating analyte profile is produced may have an indeterminate lung nodule/lesion (detected prior to production of the circulating analyte profile of the subject, e.g., by low-dose computed tomography (LDCT)), and the method may further include assessing the risk of the lung nodule being malignant (e.g., non-small cell lung cancer (NSCLC) or other malignancy) based on the circulating analyte profile of the subject. For example, the circulating analyte profile may be compared to one or more reference profiles, and based on the comparison, the risk that the indeterminate lung nodule is malignant (verses benign) may be determined. The risk assessment may be based on the circulating analyte profile being above or below a cutoff value. Thus, in certain embodiments, the subject's circulating analyte profile is indicative of the subject's lung nodule being malignant. In some embodiments, the circulating analyte profile is produced and subsequently made available to a third party, such as the subject from whom the circulating analyte profile was produced, his/her guardian or representative, a physician or health care worker, genetic counselor, or insurance agent, for example via a user interface accessible over the internet, together with an interpretation of the circulating analyte profile, e.g., in the form of a risk measure (such as an absolute risk (AR), risk ratio (RR) or odds ratio (OR)) for the nodule being malignant. The results of such risk assessment can be reported in numeric form (e.g., by risk values, such as absolute risk, relative risk, and/or an odds ratio, or by a percentage increase in risk compared with a reference), by graphical means, and/or by other means suitable to illustrate the risk to the third party.

A risk assessment may be based solely on the circulating analyte profile, or may be based in part on the circulating analyte profile. In instances where the risk assessment is based in part on the circulating analyte profile, the risk assessment may further be based on clinical parameters of the subject selected from subject age, nodule size, nodule border (spiculated or not), nodule location, subject sex, subject history of cancer, subject family history of cancer, smoking status (e.g., former versus current smoker), smoking history (including smoking intensity), and any combination thereof.

Treatment

The methods of the present disclosure may further include treating the subject for whom the circulating analyte profile is produced. In certain aspects, the subject has an indeterminant lung nodule and the methods include assessing the risk of the indeterminant lung nodule being malignant or benign. If the assessed risk of the lung nodule being malignant meets a threshold criteria, a biopsy of the nodule may be taken to diagnose the lung nodule as being malignant or benign. In some embodiments, the methods include performing such a diagnosis. If the lung nodule is diagnosed as being malignant, in some embodiments, the methods include treating the subject subsequent to the diagnosis, e.g., based on the diagnosis. The treatment may include, e.g., administering to the subject a therapeutically effective amount of a pharmaceutical agent (e.g., a chemotherapeutic agent (e.g., crizotinib, ceritinib, alectinib, brigatinib, lorlatinib, erlotinib, gefitinib, afatinib, dacomitinib, crizotinib, dabrafenib, trametinib, and/or the like), a small molecule, a biologic (e.g., an antibody), engineered cells, and/or the like), radiation therapy, and/or the like. Alternatively, or additionally, the treatment may include removing from the subject all or part of a tissue (e.g., tumor tissue) or organ that contributes to (e.g., is responsible for) the disease or condition. The treatment may include surgery to remove all or a portion of the cancer (e.g., by pneumonectomy, lobectomy, segmentectomy or wedge resection, sleeve resection, or the like); radiofrequency ablation (RFA) of all or a portion of the tumor; etc.

Devices

As summarized above, aspects of the present disclosure include sensor devices (e.g., magnetic sensor devices). The sensor devices include a panel of probes for specific binding to analytes. A sensor device of the present invention may include any of the panels of probes described hereinabove in the Methods section and in the Experimental section below of the present disclosure. According to some embodiments, the sensor devices include a panel of capture probes provided as an addressable probe array, e.g., in a sensing region of the sensor device.

According to some embodiments, a device of the present disclosure includes a panel of probes (e.g., a panel of capture probes provided as an addressable probe array) that includes probes for specific binding to two or more (e.g., 3 or more, 4 or more, 5 or more, 6 or more, 7 or more, 8 or more, 9 or more, or 10 or more) of carcinoembryonic antigen (CEA), C-X-C motif chemokine ligand 4 (CXCL4—also known as platelet factor 4 (or PF4)), C-X-C motif chemokine ligand 7 (CXCL7—also known as neutrophil activating protein 2 (or NAP2)), C-X-C motif chemokine ligand 10 (CXCL10—also known as interferon gamma-induced protein 10 (or IP10)), epidermal growth factor receptor (EGFR), pro-surfactant protein B (pro-SFTPB), tissue inhibitor of metalloproteinase 1 (TIMP1), anti-angiopoietin-like protein 3 antibody (anti-ANGPTL3), anti-14-3-3 protein theta antibody (anti-YWHAQ), anti-laminin alpha 1 antibody (anti-LAMR1), human epididymis protein 4 (HE4), anterior gradient protein 2 (AGR2), chromogranin A (CHGA), leucine-rich alpha-2-glycoprotein 1 (LRG1), anti-annexin 1 antibody (anti-ANXA1), anti-ubiquilin 1 antibody (anti-UBQLN1), interleukin 6 (IL6), interleukin 8 (IL8), C-X-C motif chemokine ligand 2 (CXCL2), C-X-C motif chemokine ligand 12 (CXCL12), C-X-C motif chemokine ligand 14 (CXCL14), defensin, beta 1 (DEFB1), fibroblast growth factor 2 (FGF2), cluster of differentiation 97 (CD97), pro-platelet basic protein (PPBP), procalcitonin (PCT), receptor for advanced glycation end products (RAGE), S100 calcium-binding protein A4 (S100A4), S100 calcium-binding protein A8 (S100A8), and osteopontin (OPN), in any desired combination.

In certain embodiments, a device of the present disclosure includes a panel of probes (e.g., a panel of capture probes provided as an addressable probe array) that includes probes for specific binding to one, two, three, or each of CEA, CXCL4, CXCL7, and CXCL10, in any desired combination. According to some embodiments, such a panel of probes further includes probes for specific binding to one, two, or each of EGFR, pro-SFTPB, and TIMP1, in any desired combination. In certain embodiments, such a panel of probes further includes one or more probes for specific binding to one or any combination of additional analytes selected from anti-ANGPTL3, anti-YWHAQ, anti-LAMR1, HE4, AGR2, CHGA, LRG1, anti-ANXA1, anti-UBQLN1, IL6, IL8, CXCL2, CXCL12, CXCL14, DEFB1, FGF2, CD97, PPBP, PCT, RAGE, S100A4, S100A8, and OPN, in any desired combination.

According to certain embodiments, the device includes a panel of probes for specific binding to from 4 to 5 analytes, from 6 to 10 analytes, from 10 to 15 analytes, from 15 to 20 analytes, from 20 to 25 analytes, from 25 to 30 analytes, from 30 to 35 analytes, from 35 to 40 analytes, from 40 to 45 analytes, from 45 to 50 analytes, from 50 to 60 analytes, from 60 to 70 analytes, from 70 to 80 analytes, from 80 to 90 analytes, from 90 to 100 analytes, from 100-200 analytes, from 200 to 300 analytes, from 300 to 400 analytes, from 400 to 500 analytes, or from 500 to 1000 analytes.

In certain embodiments, the device includes a panel of probes for specific binding to 4 or more, 5 or more, 6 or more, 7 or more, 8 or more, 9 or more, 10 or more, 15 or more, 20 or more, or 25 or more analytes. According to some embodiments, the panel of probes includes probes for specific binding to 200 or fewer analytes, 150 or fewer analytes, 125 or fewer analytes, 100 or fewer analytes, 75 or fewer analytes, 50 or fewer analytes, 40 or fewer analytes, 30 or fewer analytes, 25 or fewer analytes, 20 or fewer analytes, 15 or fewer analytes, or 10 or fewer analytes.

The panel of probes included in a sensor device of the present disclosure may further include probes for binding to circulating cells (such as circulating tumor cells (CTCs), circulating stem cells, and/or the like) and/or circulating nucleic acids (such as circulating DNA (e.g., circulating tumor DNA) and/or circulating RNA), as described hereinabove.

Magnetic Sensor Devices

According to certain embodiments, a sensor device of the present disclosure is a magnetic sensor device. Magnetic sensor devices of the present disclosure may include a magnetic sensor chip that includes a panel of probes (e.g., attached to a surface of the magnetic sensor chip), including any of the panels of the probes described elsewhere herein.

In certain aspects, the magnetic sensor chip comprises two or more magnetic sensors having capture probes attached to the surface thereof (e.g., as an addressable capture probe array). Each of the two or more magnetic sensors having capture probes attached to the surface thereof may include capture probes for binding to the same circulating analytes. Aspects of magnetic sensor devices and systems will now be described.

Magnetic Sensors

In certain aspects, a magnetic sensor device of the present disclosure includes one or more magnetic sensors. In some cases, the one or more magnetic sensors are configured to detect the presence of nearby magnetic labels without any direct physical contact between the magnetic sensor and the magnetic label. In certain embodiments, the magnetic sensors are configured to detect the presence of analytes of the two or more circulating analytes that may be present in the blood sample. For example, a magnetic label may be bound, either directly or indirectly, to an analyte, which in turn may be bound, either directly or indirectly, to the magnetic sensor. If the bound magnetic label is positioned within the detection range of the magnetic sensor, then the magnetic sensor may provide a signal indicating the presence of the bound magnetic label, and thus indicating the presence of the analyte.

In some instances, the magnetic sensors have a detection range from 1 nm to 1000 nm from the surface of the magnetic sensor, such as from 1 nm to 800 nm, including from 1 nm to 500 nm, such as from 1 nm to 300 nm, including from 1 nm to 100 nm, or from 1 nm to 75 nm, or from 1 nm to 50 nm, or from 1 nm to 25 nm, or from 1 nm to 10 nm from the surface of the magnetic sensor. In some instances, a minimization of the detection range of the sensors may facilitate detection of specifically bound analytes while minimizing detectable signals from analytes not of interest. By “detection range” is meant the distance from the surface of the magnetic sensor where the presence of a magnetic label will induce a detectable signal in the magnetic sensor. In some cases, magnetic labels positioned close enough to the surface of the magnetic sensor to be within the detection range of the magnetic sensor will induce a detectable signal in the magnetic sensor. In certain instances, magnetic labels positioned at a distance from the surface of the magnetic sensor that is greater than the detection range of the magnetic sensor will not induce a detectable or non-negligible signal in the magnetic sensor. For example, a magnetic label may have a magnetic flux that is proportional to 1/r³, where r is the distance between the magnetic sensor and the magnetic label. Thus, only those magnetic labels that are positioned in close proximity (e.g., within the detection range of the magnetic sensor) will induce a detectable signal in the magnetic sensor.

As noted, probes of the panel of probes may be bound to the surface of the magnetic sensor. For instance, a cationic polymer such as polyethyleneimine (PEI) can be used to nonspecifically bind charged probes (e.g., antibodies, antigens, ligands, nucleic acids, etc.) to the sensor surface via physiabsorption (physical absorption). Alternatively, a covalent chemistry can be used utilizing free amines or free thiol groups on the analyte-specific probe to covalently bind the analyte-specific probe to the surface of the magnetic sensor. For example, an N-hydroxysuccinimide (NHS) to 1-ethyl-3-(3-dimethylaminopropyl) carbodiimide (EDC) coupling system may be used to covalently bind the analyte-specific probe to the surface of the magnetic sensor.

In certain embodiments, the magnetic sensor is configured to generate an electrical signal in response to a magnetic label in proximity to a surface of the magnetic sensor. For example, the magnetic sensors may be configured to detect changes in the resistance of the magnetic sensor induced by changes in the local magnetic field. In some cases, binding of a magnetic label (e.g., a magnetic nanoparticle label) in close proximity to the magnetic sensor, as described above, induces a detectable change in the resistance of the magnetic sensor. For instance, in the presence of an applied external magnetic field, the magnetic labels near the magnetic sensor may be magnetized. The local magnetic field of the magnetized magnetic labels may induce a detectable change in the resistance of the underlying magnetic sensor. Thus, the presence of the magnetic labels can be detected by detecting changes in the resistance of the magnetic sensor. In certain embodiments, the magnetic sensors are configured to detect changes in resistance of 1 Ohm or less, such as 500 mOhm or less, including 100 mOhm or less, or 50 mOhm or less, or 25 mOhm or less, or 10 mOhm or less, or 5 mOhm or less, or 1 mOhm or less. In certain embodiments, the change in resistance may be expressed in parts per million (PPM) relative to the original sensor resistance, such as a change in resistance of 2 PPM or more, or 20 PPM or more, or 200 PPM or more, or 400 PPM or more, or 600 PPM or more, or 1000 PPM or more, or 2000 PPM or more, or 4000 PPM or more, or 6000 PPM or more, or 10,000 PPM or more, or 20,000 PPM or more, or 40,000 PPM or more, or 60,000 PPM or more, or 100,000 PPM or more, or 200,000 PPM or more.

The magnetic sensor may include a magnetoresistive element. Suitable magnetoresistive elements include, but are not limited to, spin valve magnetoresistive elements and magnetic tunnel junction (MTJ) magnetoresistive elements.

In certain embodiments, the magnetic sensor element is a spin valve magnetoresistive element. In certain cases, the spin valve element is a multilayer structure that includes a first ferromagnetic layer, a non-magnetic layer disposed on the first ferromagnetic layer, and a second ferromagnetic layer disposed on the non-magnetic layer. The first ferromagnetic layer may be configured to have its magnetization vector fixed in a certain direction. In some cases, the first ferromagnetic layer is called the “pinned layer”. In certain embodiments, the spin valve element includes a pinned layer with a magnetization substantially parallel to a width of the magnetic sensor element. The second ferromagnetic layer may be configured such that its magnetization vector can rotate freely under an applied magnetic field. In some cases, the second ferromagnetic layer is called the “free layer”. In some cases, the first ferromagnetic layer (which may be referred to as the “pinned layer”), is replaced by a synthetic or artificial antiferromagnet which consists of two antiparallel ferromagnetic layers separated by a nonmagnetic spacer: one of the ferromagnetic layers (which may be referred to as the “reference layer”), is underneath the non-magnetic layer which is under the “free layer”; the other ferromagnetic layer (the other “pinned layer”), is usually “pinned” by a natural antiferromagnet such as IrMn, PtMn, FeMn, or NiO.

In certain instances, the electrical resistance of a spin valve element depends on the relative orientation of the magnetization vector of the free layer to that of the pinned layer. When the two magnetization vectors are parallel, the resistance is the lowest; when the two magnetization vectors are antiparallel, the resistance is the highest. The relative change of resistance is called the magnetoresistance (MR) ratio. In certain embodiments, a spin valve element has a MR ratio of 1% to 20%, such as 3% to 15%, including 5% to 12%. In some cases, the MR ratio of a spin valve element is 10% or more in a small magnetic field, e.g., 100 Oe. Changes in the resistance of the spin valve element due to the presence of magnetic labels near the surface of the spin valve element may be detected, as described above.

In certain embodiments, the signal from the spin valve element due to the magnetic label depends on the distance between the magnetic label and the free layer of the spin valve element. In some cases, the voltage signal from a magnetic label decreases as the distance from the center of the magnetic label to the mid-plane of the free layer increases. Thus, in certain instances, the free layer in the spin valve element is positioned at the surface of the spin valve element. Positioning the free layer at the surface of the spin valve element may minimize the distance between the free layer and any bound magnetic labels, which may facilitate detection of the magnetic labels.

In certain embodiments, the spin valve element may include a passivation layer disposed on one or more of the spin valve element surfaces. In some cases, the passivation layer has a thickness of 60 nm or less, such as 50 nm or less, including 40 nm or less, 30 nm or less, 20 nm or less, 10 nm or less. For instance, the passivation layer may have a thickness of 1 nm to 10 nm, such as from 1 nm to 5 nm, including from 1 nm to 3 nm. In certain embodiments, the passivation layer includes gold, tantalum, SiO₂, Si₃N₄, combinations thereof, and the like.

In certain embodiments, the magnetic sensor element is a magnetic tunnel junction (MTJ) magnetoresistive element (also referred to herein as an MTJ element). In some cases, the MTJ element includes a multilayer structure that includes a first ferromagnetic layer, an insulating layer disposed on the first ferromagnetic layer, and a second ferromagnetic layer disposed on the insulating layer. The insulating layer may be a thin insulating tunnel barrier, and may include alumina, MgO, and the like. In some cases, electron tunneling between the first and the second ferromagnetic layers depends on the relative magnetization of the two ferromagnetic layers. For example, in certain embodiments, the tunneling current is high when the magnetization vectors of the first and second ferromagnetic layers are parallel and the tunneling current is low when the magnetization vectors of the first and second ferromagnetic layers antiparallel. In some cases, the first ferromagnetic layer may be replaced by a synthetic or artificial antiferromagnet which consists two antiparallel ferromagnetic layers separated by a nonmagnetic spacer: one of the ferromagnetic layers may be underneath the tunnel barrier; the other ferromagnetic layer may be “pinned” by a natural antiferromagnet such as IrMn, PtMn, or FeMn.

In some instances, a MTJ element has a magnetoresistance ratio (MR) of 1% to 300%, such as 10% to 250%, including 25% to 200%. Changes in the resistance of the MTJ element due to the presence of magnetic labels near the surface of the MTJ element may be detected, as described above. In some instances, the MTJ element has an MR of 50% or more, or 75% or more, or 100% or more, or 125% or more, or 150% or more, or 175% or more, or 200% or more, or 225% or more, or 250% or more, or 275% or more, or 200% or more. For instance, the MTJ element may have an MR of 225% or more.

In certain embodiments, the second ferromagnetic layer (e.g., the layer of the MTJ element positioned at the surface of the MTJ element) includes two of more layers. For example, the second ferromagnetic layer may include a first layer, a second layer disposed on the first layer, and a third layer disposed on the second layer. In some cases, the first layer is a thin ferromagnetic layer (e.g., NiFe, CoFe, CoFeB, and the like). The thin metallic layer may have a thickness of 6 nm or less, such as 5 nm or less, including 4 nm or less, 3 nm or less, 2 nm or less, or 1 nm or less, or 0.5 nm or less. The second layer may include a conductive metal, e.g., copper, aluminum, palladium, a palladium alloy, a palladium oxide, platinum, a platinum alloy, a platinum oxide, ruthenium, a ruthenium alloy, a ruthenium oxide, silver, a silver alloy, a silver oxide, tin, a tin alloy, a tin oxide, titanium, a titanium alloy, a titanium oxide, tantalum, a tantalum alloy, a tantalum oxide, combinations thereof, and the like. The second layer may have a thickness of 2 nm or less, such as 0.5 nm or less, including 0.4 nm or less, 0.3 nm or less, 0.2 nm or less, or 0.1 nm or less. The third layer may include a ferromagnetic material such as, but not limited to, NiFe, CoFe, CoFeB, and the like. The third layer may have a thickness of 6 nm or less, such as 5 nm or less, including 4 nm or less, 3 nm or less, 2 nm or less, or 1 nm or less, or 0.5 nm or less.

In some cases, the MTJ element is configured such that the distance between an associated magnetic label and the top surface of the free layer ranges from 5 nm to 1000 nm, or 10 nm to 800 nm, such as from 20 nm to 600 nm, including from 40 nm to 400 nm, such as from 60 nm to 300 nm, including from 80 nm to 250 nm.

The MTJ element may include a passivation layer disposed on one or more of the MTJ element surfaces. In some instances, the passivation layer has a thickness of 60 nm or less, such as 50 nm or less, including 40 nm or less, 30 nm or less, 20 nm or less, 10 nm or less. For example, the passivation layer may have a thickness of 1 nm to 50 nm, such as from 1 nm to 40 nm, including from 1 nm to 30 nm, or form 1 nm to 20 nm. In some instances, the passivation layer has a thickness of 30 nm. In some cases, the passivation layer includes gold, tantalum, a tantalum alloy, a tantalum oxide, aluminum, an aluminum alloy, an aluminum oxide, SiO₂, Si₃N₄, ZrO₂, combinations thereof, and the like. In certain embodiments, a passivation layer with a thickness as described above facilitates a maximization in signal detected from magnetic labels specifically bound to the sensor surface while minimizing the signal from magnetic labels that are not specifically bound.

In certain embodiments, a MTJ element has dimensions ranging from 1 μm×1 μm to 200 μm×200 μm, including dimensions of 1 μm×200 μm or less, such as 200 μm×1 μm or less, for instance 150 μm×10 μm or less, or 120 μm×5 μm or less, or 120 μm×0.8 μm or less, or 0.8 μm×120 μm or less, or 100 μm×0.7 μm or less, or 100 μm×0.6 μm or less, or 100 μm×0.5 μm or less, or 10 μm×0.6 μm or less, or 10 μm×0.5 μm or less. In some instances, a MTJ element has dimensions of 120 μm×0.8 μm or less, such as 2.0 μm×0.8 μm.

Magnetic tunnel junction (MTJ) detectors are further described in U.S. Pat. No. 9,863,939, the disclosure of which is incorporated herein by reference in its entirety for all purposes. Detectors are further described in U.S. Pat. No. 7,906,345, the disclosure of which is incorporated herein by reference in its entirety for all purposes.

In certain cases, the magnetic sensor is a multilayer thin film structure. A sensor may include alternating layers of a ferromagnetic material and a non-magnetic material. The ferromagnetic material may include, but is not limited to, Permalloy (NiFe), iron cobalt (FeCo), nickel iron cobalt (NiFeCo), CoFeB, combinations thereof, and the like. In some cases, the non-magnetic material is an noble metal, such as, but not limited to, Cu, Au, Ag, and the like. In certain embodiments, the ferromagnetic layers have a thickness of 1 nm to 10 nm, such as 2 nm to 8 nm, including 3 nm to 4 nm. In some instances, the non-magnetic layer has a thickness of 0.2 nm to 5 nm, such as 1 nm to 3 nm, including 1.5 nm to 2.5 nm, or 1.8 nm to 2.2 nm.

In certain embodiments, the magnetic sensor device may be configured to include one or more magnetic sensing areas. A magnetic sensing area may correspond to the area of the device where an array of magnetic sensors (e.g., an array of biosensors) is positioned. For instance, the magnetic sensing area may be an area on the surface of the device that is exposed to the blood sample during use, and which has an array of magnetic sensors as described above.

The magnetic sensing area may be configured to include a fluid reservoir. The fluid reservoir may be any of a variety of configurations, where the fluid reservoir is configured to hold a blood sample in contact with the magnetic sensor arrays. Accordingly, configurations of the fluid reservoirs may include, but are not limited to: cylindrical well configurations, square well configurations, rectangular well configurations, round bottom well configurations, and the like. For instance, the fluid reservoirs may include walls that separate one fluid reservoir from adjacent fluid reservoirs. The walls may be substantially vertical with respect to the surface of the reservoir plate. In some cases, the walls of each fluid reservoir define a volume of space that may receive a volume of sample equal to or less than the volume of space defined by the fluid reservoir.

In certain embodiments, a fluid reservoir has a volume of 10 mL or less, or 5 mL or less, or 3 mL or less, or 1 mL or less, such as 500 μL or less, including 100 μL or less, for example 50 μL or less, or 25 μL or less, or 10 μL or less, which is sufficient to contain a sample volume of an equal or lesser volume.

Magnetic Sensor Systems

Aspects of the present disclosure include magnetic sensor systems. In some embodiments, the magnetic sensor system includes a magnetic sensor device, and a magnetic field source. The magnetic sensor device includes a support having one or more arrays of magnetic sensors (e.g., arrays of biosensors) positioned thereon. The system may be configured to obtain signals from the one or more arrays of magnetic sensors indicating whether analytes of the circulating analytes are present in one or more corresponding blood samples.

In certain embodiments, the system includes a magnetic field source. The magnetic field source may be configured to apply a magnetic field to the magnetic sensor device (e.g., the magnetic sensor arrays) sufficient to produce a DC and/or AC field in the assay sensing area (e.g. in the area where the magnetic sensor arrays are positioned during signal acquisition). In some instances, the magnetic field source is configured to produce a magnetic field with a magnetic field strength of 1 Oe or more, or 5 Oe or more, or 10 Oe or more, or 20 Oe or more, or 30 Oe or more, or 40 Oe or more, or 50 Oe or more, or 60 Oe or more, or 70 Oe or more, or 80 Oe or more, or 90 Oe or more, or 100 Oe or more.

The magnetic field source may be positioned such that a magnetic field is produced in the area where the magnetic sensor arrays are positioned when the magnetic sensor device is in use. In some cases, the magnetic field source is configured to generate a uniform, controllable magnetic field around the set of fluid reservoirs on the reservoir plate where an assay is being performed. The magnetic field source may include one or more, such as two or more, three or more, four or more magnetic field generating components. In some cases, the magnetic field source may include one or more electromagnets, such as coil electromagnets. The coil electromagnets may include wire-wound coils. For example, the magnetic field source may include two electromagnets arranged in a Helmholtz coil geometry.

Embodiments of the systems further include computer-based systems. The systems may be configured to qualitatively and/or quantitatively assess binding interactions as described above. A “computer-based system” refers to the hardware, software, and data storage components used to analyze the signals from the magnetic sensors. The hardware of the computer-based systems may include a central processing unit (CPU), inputs, outputs, and data storage components. Any of a variety of computer-based systems is suitable for use in the subject systems. The data storage components may include any computer readable medium for recording signals from the magnetic sensor arrays, or an accessible memory component that can store signals from the magnetic sensor arrays.

To “record” data, programming or other information on a computer readable medium refers to a process for storing information, using any such methods as known in the art. Any convenient data storage structure may be chosen, depending on the method used to access the stored information. A variety of data processor programs and formats can be used for storage, e.g. word processing text file, database format, etc.

In certain embodiments, the system includes an activation and signal processing unit. The activation and signal processing unit may be configured to operably couple to the magnetic sensor device. In some instances, the activation and signal processing unit is electrically coupled to the magnetic sensor device. The activation and signal processing unit may be electrically coupled such as to provide bi-directional communication to and from the magnetic sensor device. For example, the activation and signal processing unit may be configured to provide power, activation signals, etc. to components of the magnetic sensor device, such as, but not limited to the magnetic sensor arrays. As such, the activation and signal processing unit may include an activation signal generator. The activation signal generator may be configured to provide power, activation signals, etc. to components of the analyte detection device, such as, but not limited to the magnetic sensor arrays. In some instances, the activation and signal processing unit is configured to apply a voltage across the magnetic sensor arrays ranging from 1 mV to 10 V, such as 100 mV to 5 V, including 200 mV to 1 V, for example, 300 mV to 500 mV. In some cases, the activation and signal processing unit is configured to apply a voltage across the magnetic sensor arrays of 500 mV.

Additionally, the activation and signal processing unit may be configured to receive signals from the magnetic sensor device, such as from the magnetic sensor arrays of the magnetic sensor device. The signals from the magnetic sensor(s) of the magnetic sensor device may be used to detect the presence of analytes of the two or more circulating analytes in the blood sample(s). In some instances, the activation and signal processing unit may include a processor configured to output an analyte detection result in response to receiving signals from the magnetic sensor arrays. Thus, the processor of the activation and signal processing unit may be configured to receive signals from the magnetic sensor device, process the signals according to a predetermined algorithm, obtain a result related to the presence of one or more analytes in the samples, and output the result to a user in a human-readable or an audible format. Models which may be used, e.g., to assess the risk of an indeterminant lung nodule being malignant include those described herein in the Experimental.

A “processor” references any hardware and/or software combination that will perform one or more programmed functions. For example, any processor herein may be a programmable digital microprocessor such as available in the form of an electronic controller, mainframe, server or personal computer (e.g., desktop or portable). Where the processor is programmable, suitable programming can be communicated from a remote location to the processor, or previously saved in a computer program product (such as a portable or fixed computer readable storage medium, whether magnetic, optical or solid-state device based). For example, a magnetic medium, optical disk or solid-state memory device may carry the programming, and can be read by a suitable reader communicating with the processor.

In some instances, the subject systems are configured to modulate the current applied to the magnetic sensor arrays (e.g., the sense current). The subject systems may also be configured to modulate the magnetic field generated by the magnetic field source. Modulating the sense current and the magnetic field may facilitate a minimization in signal noise, and thus a maximization in the signal to noise ratio. Additional aspects of modulating the sense current and the magnetic field are described in more detail in U.S. application Ser. No. 12/759,584, entitled “Methods and Devices for Detecting the Presence of an Analyte in a Sample, filed on Apr. 13, 2010, the disclosure of which is incorporated herein by reference in its entirety for all purposes.

Embodiments of the subject systems may also include the following components: (a) a wired or wireless communications module configured to transfer information between the system and one or more users, e.g., via a user computer, as described below; and (b) a processor for performing one or more tasks involved in the qualitative and/or quantitative analysis of the signals from the magnetic sensors. In certain embodiments, a computer program product is provided that includes a computer-usable medium having control logic (e.g., a computer software program, including program code) stored therein. The control logic, when executed by the processor of the computer, causes the processor to perform functions described herein. In other embodiments, some functions are implemented primarily in hardware using, for example, a hardware state machine. Implementation of the hardware state machine so as to perform the functions described herein may be accomplished using any convenient method and techniques.

In addition to the magnetic sensor device and activation and signal processing unit, the systems may include a number of additional components, such as, but not limited to: data output devices, e.g., monitors, speakers, etc.; data input devices, e.g., interface ports, buttons, switches, keyboards, etc.; fluid handling components, e.g., microfluidic components; power sources; power amplifiers; wired or wireless communication components; etc. For example, the systems may include fluid handling components, such as microfluidic fluid handling components. In certain embodiments, the microfluidic fluid handling components are configured to deliver a fluid to the fluid reservoirs of the reservoir plate. In some cases, the fluid includes one or more of the following: an assay composition, a blood sample, one or more detection reagents (e.g., detection antibodies, magnetic labels, and/or the like). In certain instances, the microfluidic fluid handling components are configured to deliver small volumes of fluid, such as 1 mL or less, such as 500 μL or less, including 100 μL or less, for example 50 μL or less, or 25 μL or less, or 10 μL or less.

In certain embodiments, the system is a high-sensitivity analyte detector. By “high-sensitivity” is meant that the system is configured to detect an analyte in a sample, where the concentration of the analyte in the sample is low. In some cases, the system is configured to produce a detectable signal indicating the presence of an analyte of interest in a sample where the concentration of the analyte in the sample is 1 μM or less, such as 100 nM or less, or 10 nM or less, or 1 nM or less, including 100 μM or less, or 10 μM or less, or 1 μM or less, for example 500 fM or less, or 250 fM or less, or 100 fM or less, or 50 fM or less, or 25 fM or less, such as 10 fM or less, or 5 fM or less, or 1 fM or less. Stated another way, the system may be configured to have a detection limit, e.g., a lower limit of quantitation (LLOQ), of 1 μM or less, such as 100 nM or less, or 10 nM or less, or 1 nM or less, including 100 μM or less, or 10 μM or less, or 1 μM or less, for example 500 fM or less, or 250 fM or less, or 100 fM or less, or 50 fM or less, or 25 fM or less, such as 10 fM or less, or 5 fM or less, or 1 fM or less.

In certain embodiments, the systems include a display. The display may be configured to provide a visual indication of an analyte detection result obtained from the activation and signal processing unit, as described above. The display may be configured to display a qualitative analyte detection result. For instance, the qualitative display may be configured to display qualitative indicators to a user that a sample includes or does not include a specific analyte of interest. In some embodiments, the display may be configured to display an analyte detection result, where the analyte detection result is a quantitative result, e.g., a quantitative measurement of the concentration of an analyte in a sample. For example, in embodiments where the system is configured to output a quantitative analyte detection result, the system may include a display configured to display the quantitative analyte detection result.

The magnetic sensor device optionally includes a programmable memory, which prior to and during the use of the magnetic sensor device can be programmed with relevant information such as: calibration data for each individual sensor; a record of how the biochip has been prepared with surface functionalization molecules prior to the assay; a record of all completed assay steps; a record about which sample was measured; a record of the measurement results; and the like.

Additional aspects of magnetic sensor systems are described in more detail in U.S. Pat. Nos. 9,151,763 and 9,164,100, and 9,528,995, the disclosures of which are incorporated herein by reference in their entireties for all purposes.

Kits

Also provided are kits that find use, e.g., for practicing one or more embodiments of the methods of the present disclosure.

In some embodiments, a kit of the present disclosure includes a panel of probes that includes probes for specific binding to two or more (e.g., 3 or more, 4 or more, 5 or more, 6 or more, 7 or more, 8 or more, 9 or more, or 10 or more) of carcinoembryonic antigen (CEA), C-X-C motif chemokine ligand 4 (CXCL4—also known as platelet factor 4 (or PF4)), C-X-C motif chemokine ligand 7 (CXCL7—also known as neutrophil activating protein 2 (or NAP2)), C-X-C motif chemokine ligand 10 (CXCL10—also known as interferon gamma-induced protein 10 (or IP10)), epidermal growth factor receptor (EGFR), pro-surfactant protein B (pro-SFTPB), tissue inhibitor of metalloproteinase 1 (TIMP1), anti-angiopoietin-like protein 3 antibody (anti-ANGPTL3), anti-14-3-3 protein theta antibody (anti-YWHAQ), anti-laminin alpha 1 antibody (anti-LAMR1), human epididymis protein 4 (HE4), anterior gradient protein 2 (AGR2), chromogranin A (CHGA), leucine-rich alpha-2-glycoprotein 1 (LRG1), anti-annexin 1 antibody (anti-ANXA1), anti-ubiquilin 1 antibody (anti-UBQLN1), interleukin 6 (IL6), interleukin 8 (IL8), C-X-C motif chemokine ligand 2 (CXCL2), C-X-C motif chemokine ligand 12 (CXCL12), C-X-C motif chemokine ligand 14 (CXCL14), defensin, beta 1 (DEFB1), fibroblast growth factor 2 (FGF2), cluster of differentiation 97 (CD97), pro-platelet basic protein (PPBP), procalcitonin (PCT), receptor for advanced glycation end products (RAGE), S100 calcium-binding protein A4 (S100A4), S100 calcium-binding protein A8 (S100A8), and osteopontin (OPN), in any desired combination.

In certain embodiments, a kit of the present disclosure includes a panel of probes that includes probes for specific binding to one, two, three, or each of CEA, CXCL4, CXCL7, and CXCL10, in any desired combination. According to some embodiments, such a panel of probes further includes probes for specific binding to one, two, or each of EGFR, pro-SFTPB, and TIMP1, in any desired combination. In certain embodiments, such a panel of probes further includes one or more probes for specific binding to one or any combination of additional analytes selected from anti-ANGPTL3, anti-YWHAQ, anti-LAMR1, HE4, AGR2, CHGA, LRG1, anti-ANXA1, anti-UBQLN1, IL6, IL8, CXCL2, CXCL12, CXCL14, DEFB1, FGF2, CD97, PPBP, PCT, RAGE, S100A4, S100A8, and OPN, in any desired combination.

In some embodiments, when a kit of the present disclosure includes a panel of probes as described above, the panel of probes may be a panel of capture probes provided as an addressable probe array. By way of example, a kit may include the panel of probes provided as any of the devices and systems of the present disclosure.

The subject kits may vary, and may include various devices (e.g., any of the sensor devices (e.g., magnetic sensor devises) of the present disclosure) and reagents. Reagents and devices include those mentioned herein with respect to magnetic sensor devices or components thereof (such as a magnetic sensor array), magnetic labels, one or more panels of probes, detection reagents, buffers, etc. The reagents may be provided in separate containers, such that the reagents, magnetic labels, probes, etc. may be used individually as desired. Alternatively, one or more reagents, magnetic labels, probes, etc. may be provided in the same container such that the one or more reagents, magnetic labels, capture probes, etc. is provided to a user pre-combined.

In certain embodiments, the kits include a magnetic sensor device as described above, and a magnetic label. For example, the magnetic label may be a magnetic nanoparticle, as described above. In some instances, the kits include at least reagents finding use in the methods (e.g., as described above); and a computer readable medium having a computer program stored thereon, wherein the computer program, when loaded into a computer, operates the computer to qualitatively and/or quantitatively determine binding interactions of interest from a signal (e.g., a real-time signal) obtained from a sensor (e.g., a magnetic sensor); and a physical substrate having an address from which to obtain the computer program.

A kit of the present disclosure may further include instructions. In some embodiments, the instructions include instructions for contacting a blood sample from a subject with the panel of probes to produce a circulating analyte profile of the subject. The subject kits may include instructions for practicing any of the methods of the present disclosure. In some embodiments, the instructions include instructions for contacting a blood sample from a subject from a population having a high risk of lung cancer with the panel of probes to produce a circulating analyte profile of the subject. According to certain embodiments, the instructions include instructions for contacting a blood sample from a subject who is a former or current smoker with the panel of probes to produce a circulating analyte profile of the subject. In some embodiments, the instructions include instructions for contacting a blood sample from a subject having a lung nodule (e.g., an indeterminate lung nodule (e.g., as detected by low-dose computed tomography (LDCT)) with the panel of probes to produce a circulating analyte profile of the subject. According to certain embodiments, the instructions include instructions for assessing the risk of the lung nodule being malignant based on the circulating analyte profile of the subject.

Instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., CD, DVD, Bluray, computer readable memory device (e.g., a flash memory drive), etc., on which the information has been recorded. Yet another means that may be present is a website address which may be used via the Internet to access the information at a removed site. Any convenient means may be present in the kits.

As will be appreciated from the disclosure provided above, the present disclosure has a wide variety of applications. Accordingly, the following examples are offered for illustration purposes and are not intended to be construed as a limitation on the invention in any way. Those of skill in the art will readily recognize a variety of noncritical parameters that could be changed or modified to yield essentially similar results. Thus, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

The following examples are offered by way of illustration and not by way of limitation.

### EXPERIMENTAL

### Introduction

The examples herein relate to protein biomarkers which may be measured in human blood as a characteristic associated with a malignant lung nodule in a former smoker. That information may be used alone or in combination with clinical parameters to calculate the former smoker's risk that a nodule seen on their LDCT scan is a malignant lung nodule. The protein biomarkers may be measured with the magneto-nanosensor technology developed by MagArray which overcomes the expense and low throughput of mass-spec blood protein measurement technologies and overcomes the detection limitations of ELISA based tests. Because the magneto-nanosensors are capable of multiplexing up to 80 individual detectors at one time for high-throughput, the lung nodule associated protein biomarkers can be measured at the same time with a single aliquot of patient blood. The measured levels of the protein biomarkers are then combined in a model that provides a risk of malignancy for a lung nodule. The resulting model is robust and would have clinical utility for the large population of patients undergoing lung cancer evaluation. The model can also be adapted for screening high risk populations for lung cancer and for therapy prediction and monitoring of lung cancer patients after diagnosis, either stand alone, or in conjunction with standard clinical assessments and/or other cancer biomarkers.

The principles of the magneto-nanosensor chips and giant magnetoresistive (GMR) sensors, and their use in measuring biomarkers has been described. Using that technology as configured in the MR-813 instrument system, a multiplex panel of reagents was developed to measure 3 previously reported and 5 recently identified human proteins believed to have an association with lung cancer and likely to be found in circulation. The previously reported proteins are Epidermal Growth Factor Receptor (EGFR), Pro-surfactant Protein B (pro-SFTPB), and Tissue Inhibitor of Metalloproteinases 1 (TIMP1). The 5 recently developed and tested proteins are Carcinoembryonic Antigen (CEA), Human Epididymis Protein 4 (HE4), CXCL7 (also known as Neutrophil Activating Protein 2 (NAP2)), Receptor for Advanced Glycation End-products (RAGE), and S100 calcium-binding protein A8 (S100A8).

To evaluate the clinical usefulness of those proteins, a set of over 1100 human plasma samples obtained from cohorts at 8 geographically diverse centers including Stanford University Clinic, California Pacific Medical Center, and Palo Alto Veterans Affairs Hospital, the San Francisco Veterans Affairs Medical Center, University of Pennsylvania, and the Lung Cancer Biospecimen Resource Network (Medical University of South Carolina, University of Virginia, and Washington University at St. Louis) was assembled. A subset of 405 samples was selected from the 1100 that had clinical data necessary for calculating the subject's pre-test probability of a malignant lung nodule using the Mayo Clinic risk assessment model (“Mayo model”). The 405 samples were from 3 cohorts and balanced for benign and malignant lung nodules and included current and former smokers, as shown in Table 1 and Table 2. Former smoking was defined as not smoking at the time of enrollment, while current smoking was defined as smoking up to the study enrollment. The entire cohort of former and current smokers combined is referred to as “ever smokers”.

The overall prevalence of disease in the 405-sample set was 48%, close to the target prevalence of 50% intended to ensure a balanced weighting of biomarker levels from benign and malignant disease states. A benign diagnosis is defined by two-year nodule stability or nodule resolution, and malignant diagnosis is based on the pathology report after resection or biopsy.

TABLE 1
The sample cohorts comprising the 405 subjects from which the training and testing sets were selected

| | SFVA | | | | Stanford | | | | Vanderbilt | |
|---|---|---|---|---|---|---|---|---|---|---|
| | Benign | | Malignant | | Benign | | Malignant | | Benign | |
| | (n = 67) | | (n = 14) | | (n = 57) | | (n = 71) | | (n = 81) | |
| Age (years) | | | | | | | | | | |
| Mean (SD) | 68.8 | (7.33) | 71.9 | (6.23) | 62 | (11.6) | 70.8 | (8.89) | 57.6 | (11.2) |
| Median [Min, Max] | 70 | [52, 87] | 72 | [58, 83] | 64 | [25, 85] | 70 | [46, 89] | 59 | [33, 84] |
| Sex | | | | | | | | | | |
| Female | 1 | (1.5%) | 1 | (7.1%) | 14 | (24.6%) | 16 | (22.5%) | 33 | (40.7%) |
| Male | 66 | (98.5%) | 13 | (92.9%) | 43 | (75.4%) | 55 | (77.5%) | 48 | (59.3%) |
| Pack Years | | | | | | | | | | |
| Mean (SD) | 50.8 | (41.9) | 36.5 | (30.8) | 43.1 | (41.4) | 48 | (30.6) | 44.2 | (32) |
| Median [Min, Max] | 40 | [0, 210] | 25.3 | [6, 120] | 30 | [0, 200] | 45 | [1.5, 150] | 40 | [0, 200] |
| Missing | 0 | (0%) | 0 | (0%) | 22 | (38.6%) | 11 | (15.5%) | 3 | (3.7%) |
| Nodule Size (mm) | | | | | | | | | | |
| Mean (SD) | 9.96 | (4.49) | 16.1 | (6.53) | 14.9 | (6.14) | 18.8 | (6.19) | 15.2 | (6.43) |
| Median [Min, Max] | 8 | [4, 22] | 14 | [7, 29] | 15 | [6, 30] | 20 | [8, 30] | 14 | [4, 28] |
| Nodule Location | | | | | | | | | | |
| Lower or Mid Lobe | 41 | (61.2%) | 8 | (57.1%) | 29 | (50.9%) | 32 | (45.1%) | 37 | (45.7%) |
| Upper Lobe | 26 | (38.8%) | 6 | (42.9%) | 28 | (49.1%) | 39 | (54.9%) | 44 | (54.3%) |
| Histology | | | | | | | | | | |
| Benign Nodule | 67 | (100%) | 0 | (0%) | 57 | (100%) | 0 | (0%) | 61 | (75.3%) |
| Benign Granuloma | 0 | (0%) | 0 | (0%) | 0 | (0%) | 0 | (0%) | 20 | (24.7%) |
| Adenocarcinoma | 0 | (0%) | 12 | (85.7%) | 0 | (0%) | 58 | (81.7%) | 0 | (0%) |
| Squamous Cell Carcinoma | 0 | (0%) | 2 | (14.3%) | 0 | (0%) | 8 | (11.3%) | 0 | (0%) |
| NSCLC | 0 | (0%) | 0 | (0%) | 0 | (0%) | 5 | (7%) | 0 | (0%) |
| Other | 0 | (0%) | 0 | (0%) | 0 | (0%) | 0 | (0%) | 0 | (0%) |
| Stage | | | | | | | | | | |
| Stage I or II | 0 | (0%) | 0 | (0%) | 0 | (0%) | 58 | (81.7%) | 0 | (0%) |
| Stage III or IV | 0 | (0%) | 0 | (0%) | 0 | (0%) | 13 | (18.3%) | 0 | (0%) |
| Not Determined | 0 | (0%) | 14 | (100%) | 0 | (0%) | 0 | (0%) | 0 | (0%) |
| Missing | 67 | (100%) | 0 | (0%) | 57 | (100%) | 0 | (0%) | 81 | (100%) |
| Race/Ethnicity | | | | | | | | | | |
| Asian | 2 | (3%) | 0 | (0%) | 3 | (5.3%) | 7 | (9.9%) | 0 | (0%) |
| Black | 8 | (11.9%) | 3 | (21.4%) | 2 | (3.5%) | 3 | (4.2%) | 3 | (3.7%) |
| White | 56 | (83.6%) | 10 | (71.4%) | 48 | (84.2%) | 57 | (80.3%) | 78 | (96.3%) |
| Latino | 0 | (0%) | 0 | (0%) | 2 | (3.5%) | 1 | (1.4%) | 0 | (0%) |
| Other | 0 | (0%) | 0 | (0%) | 2 | (3.5%) | 3 | (4.2%) | 0 | (0%) |
| Missing | 1 | (1.5%) | 1 | (7.1%) | 0 | (0%) | 0 | (0%) | 0 | (0%) |

| | Vanderbilt | | Overall | | | |
|---|---|---|---|---|---|---|
| | Malignant | | Benign | | Malignant | |
| | (n = 108) | | (n = 205) | | (n = 193) | |
| Age (years) | | | | | | |
| Mean (SD) | 66.4 | (8.92) | 62.5 | (11.2) | 68.4 | (9) |
| Median [Min, Max] | 66.5 | [49, 89] | 64 | [25, 87] | 69 | [46, 89] |
| Sex | | | | | | |
| Female | 41 | (38%) | 48 | (23.4%) | 58 | (30.1%) |
| Male | 67 | (62%) | 157 | (76.6%) | 135 | (69.9%) |
| Pack Years | | | | | | |
| Mean (SD) | 53 | (34.1) | 46.5 | (37.7) | 50.1 | (32.9) |
| Median [Min, Max] | 49.5 | [1.25, 200] | 40 | [0, 210] | 47 | [1.25, 200] |
| Missing | 0 | (0%) | 25 | (12.2%) | 11 | (5.7%) |
| Nodule Size (mm) | | | | | | |
| Mean (SD) | 17.4 | (5.64) | 13.4 | (6.25) | 17.8 | (5.94) |
| Median [Min, Max] | 17 | [7, 29] | 12.1 | [4, 30] | 17 | [7, 30] |
| Nodule Location | | | | | | |
| Lower or Mid Lobe | 50 | (46.3%) | 107 | (52.2%) | 90 | (46.6%) |
| Upper Lobe | 58 | (53.7%) | 98 | (47.8%) | 103 | (53.4%) |
| Histology | | | | | | |
| Benign Nodule | 0 | (0%) | 185 | (90.2%) | 0 | (0%) |
| Benign Granuloma | 0 | (0%) | 20 | (9.8%) | 0 | (0%) |
| Adenocarcinoma | 70 | (64.8%) | 0 | (0%) | 140 | (72.5%) |
| Squamous Cell Carcinoma | 23 | (21.3%) | 0 | (0%) | 33 | (17.1%) |
| NSCLC | 7 | (6.5%) | 0 | (0%) | 12 | (6.2%) |
| Other | 8 | (7.4%) | 0 | (0%) | 8 | (4.1%) |
| Stage | | | | | | |
| Stage I or II | 105 | (97.2%) | 0 | (0%) | 163 | (84.5%) |
| Stage III or IV | 1 | (0.9%) | 0 | (0%) | 14 | (7.3%) |
| Not Determined | 0 | (0%) | 0 | (0%) | 14 | (7.3%) |
| Missing | 2 | (1.9%) | 205 | (100%) | 2 | (1%) |
| Race/Ethnicity | | | | | | |
| Asian | 0 | (0%) | 5 | (2.4%) | 7 | (3.6%) |
| Black | 6 | (5.6%) | 13 | (6.3%) | 12 | (6.2%) |
| White | 100 | (92.6%) | 182 | (88.8%) | 167 | (86.5%) |
| Latino | 0 | (0%) | 2 | (1%) | 1 | (0.5%) |
| Other | 2 | (1.9%) | 2 | (1%) | 5 | (2.6%) |
| Missing | 0 | (0%) | 1 | (0.5%) | 1 | (0.5%) |

TABLE 2
Smoking History of the Cohorts in the 405 Subject Set

| | SFVA | Stanford | Vanderbilt | Total |
|---|---|---|---|---|
| Benign | | | | |
| Current Smokers | 22 | 25 | 44 | 91 |
| Former Smokers | 45 | 32 | 41 | 118 |
| Ever Smokers | 67 | 57 | 85 | 209 |
| Malignant | | | | |
| Current Smokers | 6 | 21 | 49 | 76 |
| Former Smokers | 14 | 50 | 56 | 120 |
| Ever Smokers | 20 | 71 | 105 | 196 |
| All | | | | |
| Current Smokers | 28 | 46 | 93 | 167 |
| Former Smokers | 59 | 82 | 97 | 238 |
| Ever Smokers | 87 | 128 | 190 | 405 |

Overall disease Prevalence: 48%;
Overall Current Smokers: 41%

Levels of the 8 proteins were obtained for the 405 samples using the MR813 multiplexed panel of assays. The samples were run in two studies with 150 samples in the first study and 255 in the second study. The samples for the first study were selected to cover the range of pre-test probabilities of malignancy as determined with the Mayo Clinic model. The second study contained the remaining 255 samples. In both studies, the samples were run in a randomized order by technicians who were blinded to the sample clinical information. The data collection and analysis were done according to sound biomarker study design principles.

The statistical analysis of the assay results began by assessing the assay coefficients of variation (CV) between replicate measurements as an indicator of the test reproducibility. The assay data exhibited within run variability of 10% or less and the overall variability of an on-board run control, that was run with each set of assays, was generally less than 15%. Assay replicates that exhibited more than a 30% CV were repeated. There were 12 samples repeated in the first study and 15 samples in the second study for a total of less than 7% repeats due to unexpectedly large % CV.

The assay data were analyzed as raw GMR parts per million (PPM) signals and also normalized as a ratio of the sample signal to the signal of the run control obtained for every 3 samples.

Several pre-specified analyses were performed to determine the diagnostic accuracy of the panel to differentiate malignant from benign disease, using logistic regression modeling techniques with cross-validation to generate accuracy, sensitivity, specificity, NPV, and PPV metrics. The cross-validation techniques allowed the estimation of how much the results would vary if the model were applied to other possible cohorts. Such techniques reduce the false discovery rate when defining the model components and help ensure the coefficients of the logistic regression algorithm are not overly specific for just the training set.

### Example 1—Prediction of Lung Cancer Risk in Former Smokers

Tested was the K₂EDTA plasma from a 405-patient cohort with a lung nodule on CT scan as a case-control retrospective design collected from three medical centers. Cases were diagnosed with a lung cancer from their pathology report and controls were subjects with a negative/normal pathology or stable nodules for 2 years. Magneto-nanosensors and sandwich immunoassays developed at Stanford and MagArray were used to measure the protein biomarkers Epidermal Growth Factor Receptor (EGFR), Pro-surfactant Protein B (pro-SFTPB), Tissue Inhibitor of Metalloproteinases 1 (TIMP1), Carcinoembryonic Antigen (CEA), Human Epididymis Protein 4 (HE4), Neutrophil Activating Protein 2 (NAP2), Receptor for Advanced Glycation End-products (RAGE), and S100 calcium-binding protein A8 (S100A8) in 20 μL of subject plasma. The levels of protein biomarkers were then analyzed in subcohorts of those patients stratified by diagnosis and smoking status to understand the relationship of the biomarkers to diagnosis and smoking status using ANOVA and logistic regression.

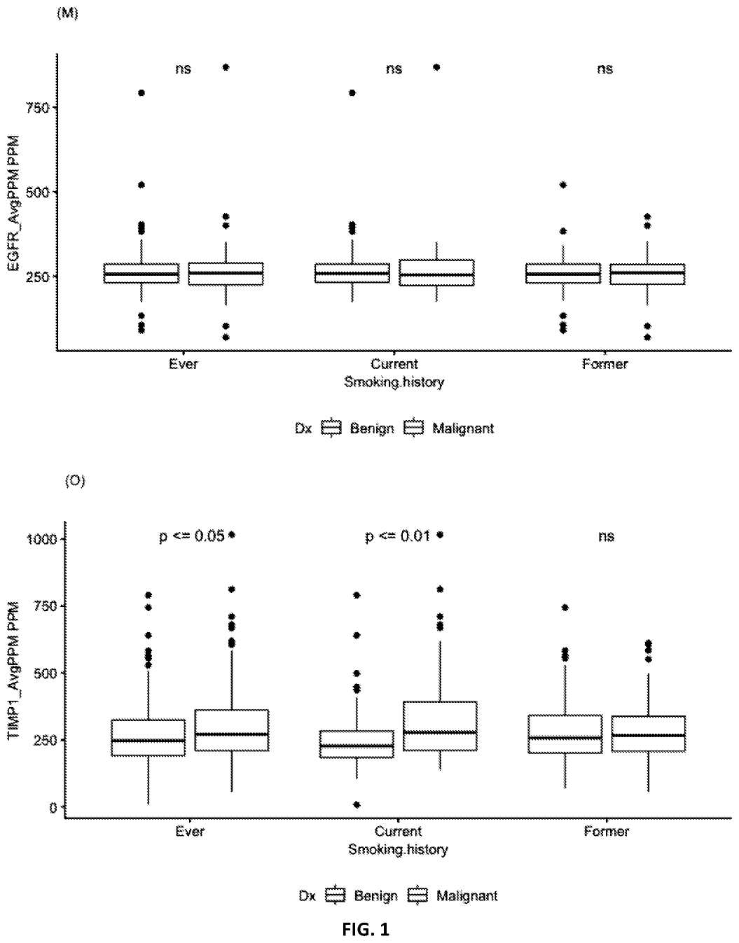

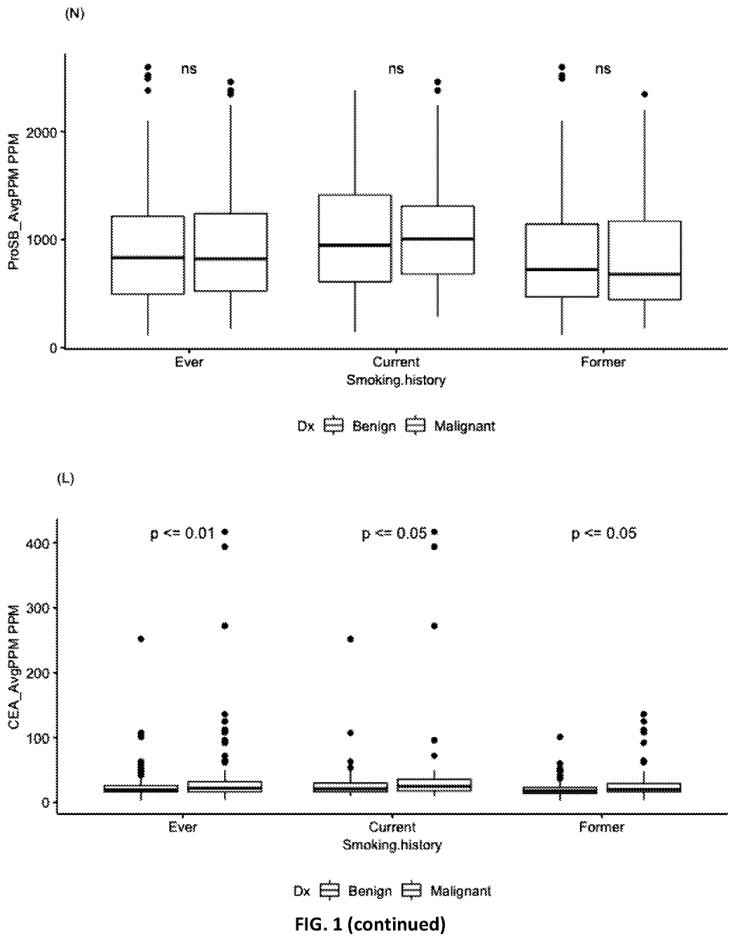

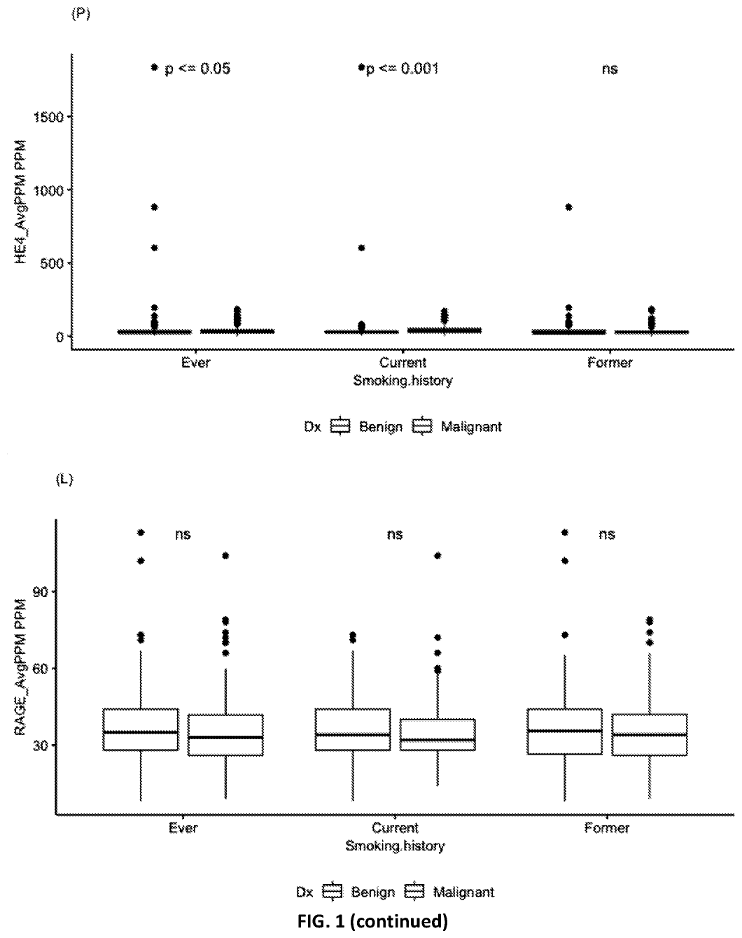

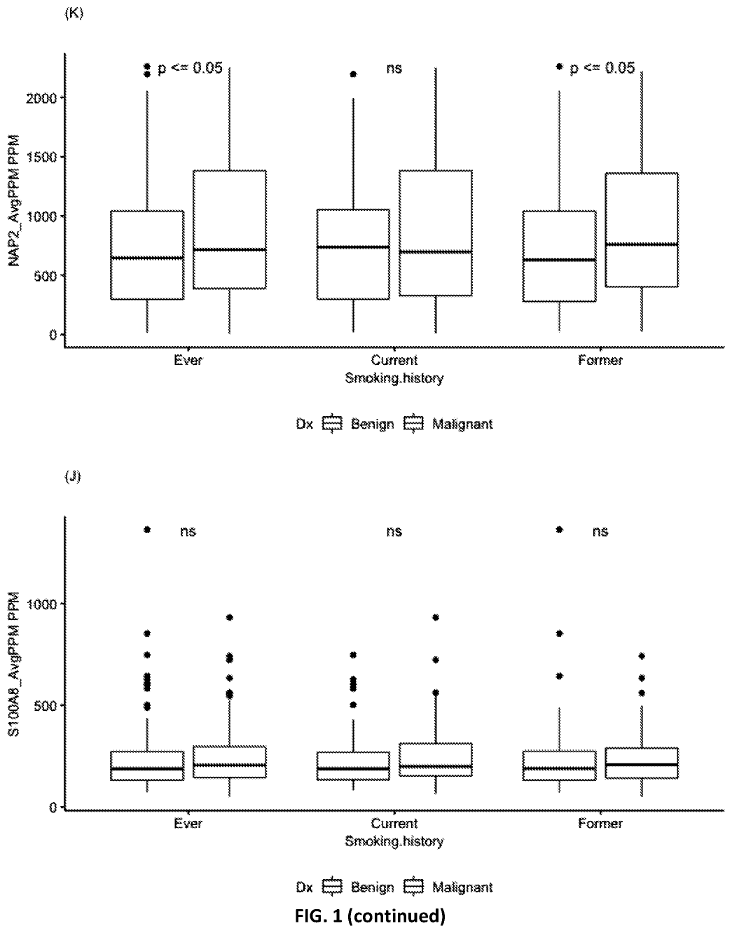

The average levels of the eight biomarkers in the subjects stratified by smoking status are shown in FIG. 1. Significant differences in levels between benign and malignant diagnoses are indicated by a p-value less than or equal to 0.05. P-values greater than 0.05 are not significant and indicated by “ns”.

The 209 benign and 196 malignant diagnosis samples comprising the 405-sample set were each randomly split into a 2/3 and 1/3 subset for training of models with different samples than are used to test the models. This was done to reduce the likelihood of overly optimistic test performance of a model that can occur when a model is tested on the same data set used to train it.

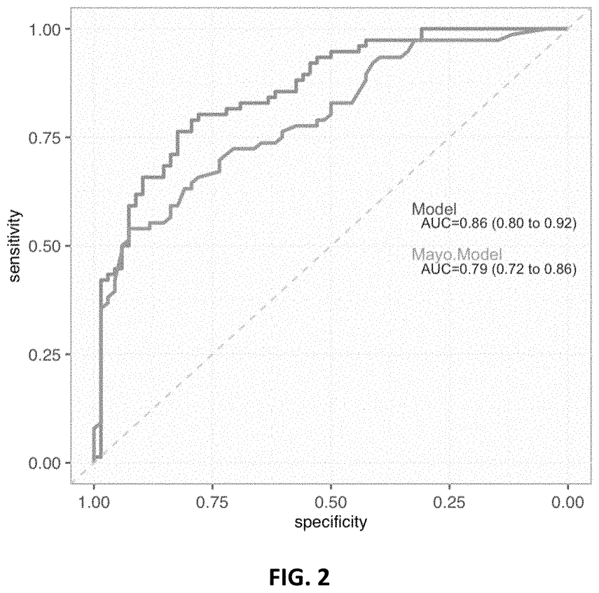

Using the biomarker levels plus the subject clinical data with the former smokers, a generalized linear modeling process was used to evaluate different variable combinations in a training set using the 1/3 samples subset. A model was identified that was able to distinguish within the test subset (2/3 samples) with malignant disease from those with benign disease with an accuracy of 79%. That model, designated as #217-3092, consisted of the biomarkers CEA, EGFR, NAP2, ProSB, and TIMP1 with the clinical factors subject age, nodule size, subject sex, and nodule border (spiculated or not). The receiver operating characteristic (ROC) curve for model 217-3092 is shown in FIG. 2 with an area under the curve (AUC) of 0.86 compared to the Mayo model AUC for the same samples of 0.79.

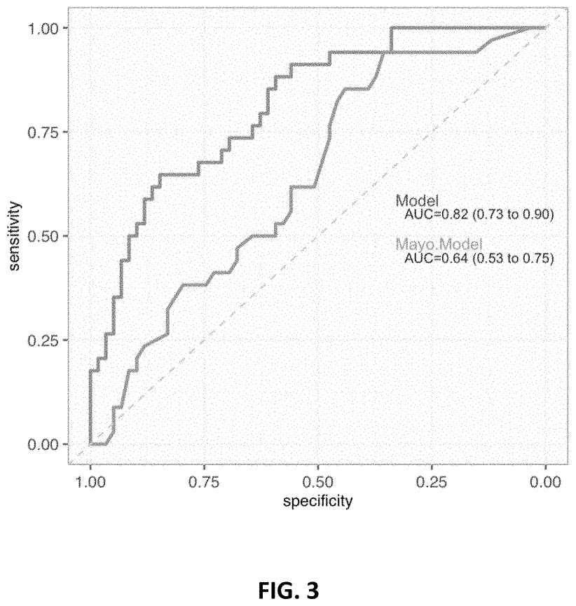

Exploring the model 217_3092 performance in those 93 former smoking subjects given a pre-test probability of malignancy by the Mayo model between 0.05 and 0.65 (the intermediate risk range as defined in published guidelines) shows the biomarker and clinical factor combination provides an improved AUC compared to the Mayo model (FIG. 3). Model 217_3092 performance summaries are shown in Table 3 where the indicators of model performance estimate a negative predictive value (NPV) of 91% with a respectable 51% positive predictive value (PPV) given a 0.25 prevalence of disease. That level of disease is based on a study of community pulmonologists where 1 in 4 people seeking their care were diagnosed with malignant lung cancer. Other important measures of clinical performance show Model 217_3092 exhibits excellent sensitivity (76%) and specificity (82%) at a cutoff of 0.485. Finally, the ability of the model to accurately classify Mayo model intermediate risk subjects was considered by using metric of net reclassification that determines the net number of subjects correctly classified after subtracting out those incorrectly diagnosed by the model. The percent reclassification of malignant subjects in the Mayo model intermediate risk category (IDm_RI) was 6%, while the number of intermediate risk benign subjects reclassified (IDb_RI) was 48%, giving an overall net reclassification index (ID_NetRI) of 55%.

TABLE 1
Summary of model 217_3092 test set performance in former smokers

| | | Mayo | AUC Diff | optimal | | | | | | | | | |
|---|---|---|---|---|---|---|---|---|---|---|---|---|---|
| Model | AUC | AUC | v Mayo | cutoff | Accuracy | Sensitivity | Specificity | prevalence | NPV | PPV | IDm_RI | IDb_RI | ID_NetRI |
| 217_3092 | 0.86 | 0.79 | 0.07 | 0.485 | 79% | 76% | 82% | 0.25 | 91% | 59% | 6% | 48% | 55% |

### Example 2—Prediction of Lung Cancer Risk in Current Smokers

Evaluated in this example was the discriminatory ability of the biomarkers plus clinical factors combination of model 217_3092 (biomarkers: CEA, EGFR, NAP2, ProSB, and TIMP1 with the clinical factors subject age, nodule size, subject sex, and nodule border (spiculated or not) to predict malignancy in the current smoker subset using generalized logistic regression prediction methods. The training set consisted of the 2/3 cohort while the test set was the remaining 1/3 cohort of the current smokers from the 405-sample set.

The overall performance of the model is summarized in Table 4 where the accuracy of 69% and sensitivity of 61% with a 77% specificity are using a cutoff of 0.508.

More significantly is the reclassification performance where a net 41% of the current smokers labeled as intermediate risk (ID_NetRI) are then correct called benign or malignant. That is the sum of 6% net malignant intermediate risk current smokers by the Mayo model (IDM_RI) and 35% net benign intermediate risk current smokers (IDb_RI).

TABLE 2
Summary of model 217_3092 test performance in current smokers

| | | Mayo | AUC Diff | optimal | | | | | | | | | |
|---|---|---|---|---|---|---|---|---|---|---|---|---|---|
| Model | AUC | AUC | v Mayo | cutoff | Accuracy | Sensitivity | Specificity | prevalence | NPV | PPV | IDm_RI | IDb_RI | ID_NetRI |
| 217_3092 | 0.75 | 0.72 | 0.03 | 0.508 | 69% | 61% | 77% | 0.25 | 86% | 47% | 6% | 35% | 41% |

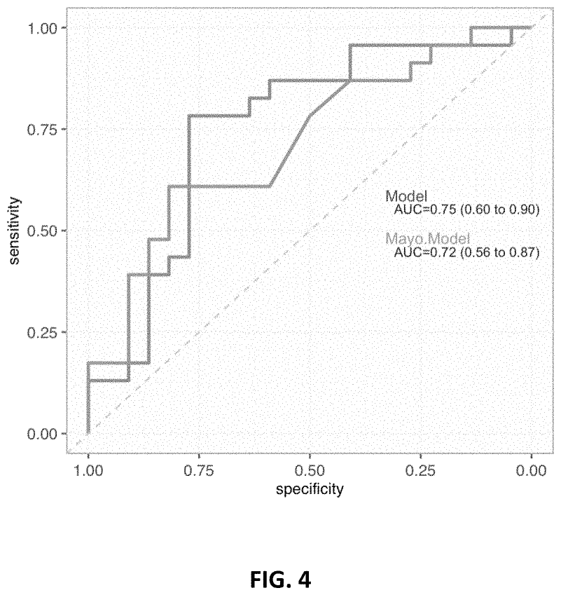

The model performance as measured by ROC curve AUC was 0.75 compared to the Mayo model of 0.72 (FIG. 4) considering the entire testing cohort of current smokers.

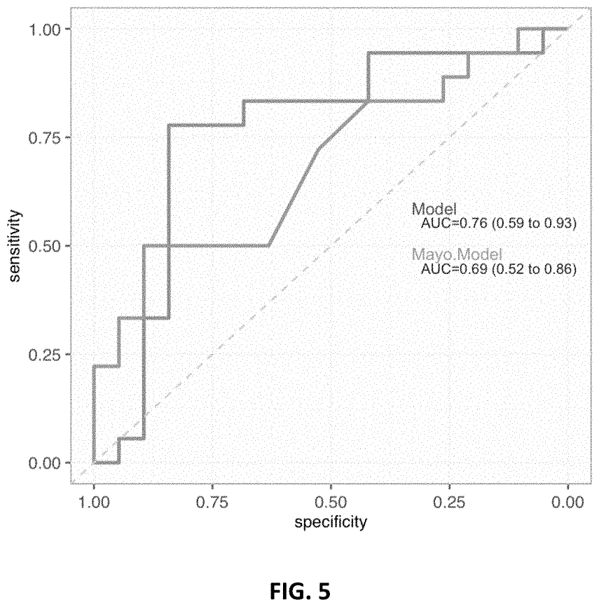

Evaluating the Mayo risk score intermediate risk current smoker subjects (n=37) with the model 217_3092 shows improved performance compared to the mayo model itself (FIG. 5). The model AUC=0.76 compared to an AUC=0.69 with the Mayo model.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

1. A method of producing a circulating analyte profile of a subject, comprising:
contacting a blood sample from a subject with a panel of probes for specific binding to analytes comprising:
two or more of carcinoembryonic antigen (CEA), C-X-C motif chemokine ligand 4 (CXCL4), C-X-C motif chemokine ligand 7 (CXCL7), and C-X-C motif chemokine ligand 10 (CXCL10); and
one or more of epidermal growth factor receptor (EGFR), pro-surfactant protein B (pro-SFTPB), and tissue inhibitor of metalloproteinase 1 (TIMP1); and
detecting the presence or absence of binding of the analytes to probes of the panel of probes, to produce a circulating analyte profile of the subject.

2. The method according to claim 1, wherein the blood sample is contacted with a panel of probes for specific binding to analytes comprising three or each of CEA, CXCL4, CXCL7, and CXCL10.

3. The method according to claim 1, wherein the blood sample is contacted with a panel of probes for specific binding to analytes comprising CEA, CXCL4, CXCL7, and CXCL10.

4. The method according to claim 1, wherein the blood sample is contacted with a panel of probes for specific binding to analytes comprising two or each of EGFR, pro-SFTPB, and TIMP1.

5. The method according to claim 1, wherein the blood sample is contacted with a panel of probes for specific binding to analytes comprising EGFR, pro-SFTPB, and TIMP1.

6. The method according to claim 1, wherein the panel of probes further comprises one or more probes for specific binding to one or any combination of additional analytes selected from the group consisting of:
anti-angiopoietin-like protein 3 antibody (anti-ANGPTL3), anti-14-3-3 protein theta antibody (anti-YWHAQ), anti-laminin alpha 1 antibody (anti-LAMR1), human epididymis protein 4 (HE4), anterior gradient protein 2 (AGR2), chromogranin A (CHGA), leucine-rich alpha-2-glycoprotein 1 (LRG1), anti-annexin 1 antibody (anti-ANXA1), anti-ubiquilin 1 antibody (anti-UB QLN1), interleukin 6 (IL6), interleukin 8 (IL8), C-X-C motif chemokine ligand 2 (CXCL2), C-X-C motif chemokine ligand 12 (CXCL12), C-X-C motif chemokine ligand 14 (CXCL14), defensin, beta 1 (DEFB1), fibroblast growth factor 2 (FGF2), cluster of differentiation 97 (CD97), pro-platelet basic protein (PPBP), procalcitonin (PCT), receptor for advanced glycation end products (RAGE), S100 calcium-binding protein A4 (S100A4), S100 calcium-binding protein A8 (S100A8), and osteopontin (OPN),
wherein the method further comprises detecting the presence or absence of binding of the one or any combination of additional analytes to probes of the panel of probes to produce the circulating analyte profile of the subject.

7. The method according to claim 1, wherein the panel of probes comprises probes for binding to 4 or more, 5 or more, 6 or more, 7 or more, 8 or more, 9 or more, 10 or more, 15 or more, 20 or more, or 25 or more analytes.

8. The method according to claim 1, wherein the panel of probes comprises probes for specifically binding to 200 or fewer analytes, 150 or fewer analytes, 125 or fewer analytes, 100 or fewer analytes, 75 or fewer analytes, 50 or fewer analytes, 40 or fewer analytes, 30 or fewer analytes, or 25 or fewer analytes.

9. The method according to claim 1, wherein detecting the presence or absence of binding of the analytes comprises quantifying detected analytes.

10. The method according to claim 1, wherein the panel of probes further comprises probes for binding to circulating tumor cells, wherein the method further comprises detecting the presence or absence of binding of the circulating tumor cells to probes of the panel of probes to produce the circulating analyte profile of the subject.

11. The method according to claim 10, wherein detecting the presence or absence of binding of the circulating tumor cells comprises quantifying detected circulating tumor cells.

12. The method according to claim 1, wherein the panel of probes further comprises probes for binding to tumor DNA, wherein the method further comprises detecting the presence or absence of binding of tumor DNA to probes of the panel of probes to produce the circulating analyte profile of the subject.

13. The method according to claim 12, wherein detecting the presence or absence of binding of tumor DNA comprises quantifying detected tumor DNA.

14. The method according to claim 1, wherein the subject is from a population having a high risk of lung cancer.

15. The method according to claim 14, wherein the subject is a former or current smoker.

16. The method according to claim 15, wherein the former or current smoker has a lung nodule.

17. The method according to claim 16, wherein the former or current smoker has a lung nodule detected by low-dose computed tomography (LDCT).

18. The method according to claim 17, further comprising assessing the risk of the lung nodule being malignant based on the circulating analyte profile of the subject.

19. The method according to claim 18, wherein the assessing is further based on one or any combination of clinical parameters of the subject selected from the group consisting of: subject age, nodule size, subject sex, nodule border (spiculated or not), nodule location, subject history of cancer, subject family history of cancer, and smoking history (including smoking intensity).

20. The method according to claim 18, comprising assessing the risk of the lung nodule being non-small cell lung cancer (NSCLC).

21. The method according to claim 1 20, wherein the blood sample is a whole blood sample, a plasma sample, or a serum sample.

22. The method according to claim 1, wherein the panel of probes is a panel of capture probes provided as an addressable probe array.

23. The method according to claim 22, wherein the addressable probe array is present on a magnetic sensor chip of a magnetic sensor device.

24. The method according to claim 23, wherein the magnetic sensor chip comprises two or more magnetic sensors having capture probes attached to the surface thereof.

25. The method according to claim 24, wherein each of the two or more magnetic sensors having capture probes attached to the surface thereof comprises capture probes for binding to the same analytes.

26. The method according to claim 24, wherein each magnetic sensor comprises a magnetoresistive element.

27. The method according to claim 26, wherein the magnetoresistive element is a spin valve magnetoresistive element or a magnetic tunnel junction (MTJ) magnetoresistive element.

28. The method according to claim 27, wherein detecting the presence of binding of the analytes to probes of the panel of probes comprises detecting a magnetically-labeled detection reagent bound to a captured analyte.

29. The method according to claim 28, wherein the magnetically-labeled detection reagent is bound indirectly to the captured analyte.

30. The method according to claim 29, wherein the magnetically-labeled detection reagent is part of a complex comprising the capture probe, the analyte, a primary detection reagent specifically bound to the analyte, and the magnetically-labeled detection reagent bound to the primary detection reagent.

31. The method according to claim 30, wherein detecting the presence of binding of the analytes to probes of the panel of probes comprises detecting a resistance change in the magnetoresistive element induced by the magnetically-labeled detection reagent.

32. A sensor device, comprising:
a panel of capture probes provided as an addressable probe array, wherein the panel comprises probes for specific binding to analytes comprising:
two or more of carcinoembryonic antigen (CEA), C-X-C motif chemokine ligand 4 (CXCL4), C-X-C motif chemokine ligand 7 (CXCL7), and C-X-C motif chemokine ligand 10 (CXCL10); and
one or more of epidermal growth factor receptor (EGFR), pro-surfactant protein B (pro-SFTPB), and tissue inhibitor of metalloproteinase 1 (TIMP1).

33.-47. (canceled)

48. A kit comprising:
a panel of probes for specific binding to analytes comprising:
two or more of carcinoembryonic antigen (CEA), C-X-C motif chemokine ligand 4 (CXCL4), C-X-C motif chemokine ligand 7 (CXCL7), and C-X-C motif chemokine ligand 10 (CXCL10); and
one or more of epidermal growth factor receptor (EGFR), pro-surfactant protein B (pro-SFTPB), and tissue inhibitor of metalloproteinase 1 (TIMP1); and
instructions for contacting a blood sample from a subject with the panel of probes to produce a circulating analyte profile of the subject.

49.-61. (canceled)
